(19) United States
(12) Patent Application Publication
(10) Pub. No.: US 2023/0248744 A1
(43) Pub. Date: Aug. 10, 2023

(54) 25-HYDROXYVITAMIN D2 AND/OR D3 FOR USE IN OBESITY

(71) Applicants: CARBOGEN AMCIS B.V., Veenendaal (NL); AAMANYA AG, Vitznau (CH)

(72) Inventors: Peter M. MÜLLER, Therwil (CH); Michael F. HOLICK, Boston, MA (US)

(73) Assignees: CARBOGEN AMCIS B.V., Veenendaal (NL); AAMANYA AG, Vitznau (CH)

(21) Appl. No.: 17/917,498

(22) PCT Filed: Apr. 6, 2021
(86) PCT No.: PCT/EP2021/058958

§ 371 Date: Oct. 6, 2022

(30) Foreign Application Priority Data
Apr. 7, 2020 (EP) 20168450.3
Apr. 20, 2020 (EP) 20170350.1

(51) Int. Cl. A61K 31/592; A61K 31/593; A61P 3/04; A61P 3/02

(52) U.S. Cl. CPC A61K 31/592; A61K 31/593; A61P 3/04; A61P 3/02

(57) ABSTRACT

The present invention relates to the field of vitamin D insufficiency and deficiency, viz. too low blood levels of 25(OH)D, and to their prevention and treatment. It provides a vitamin D supplement which comprises (i) a combination of 25(OH)D3 and/or 25(OH)D2 and vitamin D2; or (ii) a combination of 25(OH)D3 and/or 25(OH)D2 and vitamin D3; or (iii) a combination of 25(OH)D3 and/or 25(OH)D2 and a combination of vitamin D2 and vitamin D3, for use in the treatment of and/or prophylaxis of vitamin D insufficiency or deficiency in obese persons, overweight persons having the tendency to get obese or persons having a history of malabsorption of vitamin D or persons with a genetic disposition to develop obesity. Also provided are corresponding compositions and dosage regimens comprising such vitamin D supplements as well as kits comprising such compositions. Furthermore, corresponding dosage regimens for daily administration and corresponding product package inserts are also provided. The invention also relates to corresponding methods of treatment.

The present invention relates to the field of vitamin D and the prevention and/or treatment of vitamin D insufficiency and/or deficiency in obese persons, persons having the tendency to get obese and in persons having a history of malabsorption of vitamin D. There are primarily two forms of vitamin D, viz. vitamin D2 and vitamin D3. In the bloodstream vitamin D2 is transformed (metabolized) in the liver to 25-hydroxyvitamin D2 (abbreviated 25(OH)D2; also called ercalcidiol), whereas vitamin D3 is transformed (metabolized) in the liver to 25-hydroxyvitamin D3 (abbreviated 25(OH)D3; also called calcifediol). When hereinbelow the term “vitamin D” is used, then either vitamin D2 or vitamin D3 is meant, or a combination of vitamin D2 and vitamin D3. When hereinbelow the term “25(OH)D” is used, then either the vitamin D2 metabolite 25(OH)D2 or the vitamin D3 metabolite 25(OH)D3 is meant, or a combination of the vitamin D metabolites 25(OH)D2 and 25(OH)D3.

The D vitamins are for several reasons special in comparison to other vitamins. A main point is that they do not have to be exclusively taken up from the diet, but that one of them (vitamin D3) is in part endogenously formed, viz. generated in the skin upon exposure to UV light (mainly solar radiation) from a biosynthetic precursor of cholesterol. This part of the organism's delivery of vitamin D may be defined as endogenous vitamin D in the narrow sense. A broader definition is based on the following consideration: Vitamin D has to undergo two metabolic steps to exert its classical activities, in particular the avoidance of rickets and of osteomalacia. 25(OH)D is the relevant first metabolite in this activating sequence and is the vitamin's main storage form in blood. It is, besides some uptake from the diet (or supplementation), formed from vitamin D which is released from endogenous stores (mainly fatty tissues). Accordingly, a broader definition of endogenous vitamin D includes vitamin D3 which is formed by UV light in skin and vitamin D (viz. D2 and D3) which is released from endogenous storage places to be metabolized. Finally, in the broadest sense endogenous vitamin D includes vitamin D3 formed by UV light in skin, vitamin D which is released from endogenous stores, and vitamin D which is present in endogenous stores. Levels of vitamin D in this broadest sense may be called adequate, if they sustain a healthy homeostasis overall, and it will be outlined in this document that there are circumstances (e.g. in obesity) under which the adequate level of vitamin D should not be defined as the level which is per se ensuring the broadly accepted target of blood levels of 25(OH)D≥30 ng/mL.

More particularly the invention relates to a method of preventing or treating a vitamin D insufficiency or deficiency in obese persons, persons having the tendency to get obese and in persons having a history of malabsorption of vitamin D with a vitamin D supplement. The vitamin D insufficiency or deficiency is herein defined as characterized by too low or much too low blood level of 25(OH)D, respectively. The vitamin D supplement viz. a medicament, a nutraceutical or a food additive comprises either 25(OH)D3 or 25(OH)D2; or a combination of 25(OH)D3 and 25(OH)D2, provided the person has a sufficient level of endogeneous vitamin D; or a combination of 25(OH)D3 and/or 25(OH)D2 and vitamin D2, or a combination of 25(OH)D3 and/or 25(OH)D2 and vitamin D3, or a combination of 25(OH)D3 and/or 25(OH)D2 and a combination of vitamin D3 and vitamin D2. Preferably the ratio of the 25 hydroxy form of the vitamin D to vitamin D is in the range between 1 to 5 and 5 to 1, preferentially between 1 to 2 and 2 to 1, or in a ratio of 1 to 1. Also provided are corresponding methods of treatment, compositions and kits comprising said compositions.

### BACKGROUND OF THE INVENTION

Vitamin D stands for a family of nutrients that share similarities in chemical structure. The most commonly found members are vitamin D2 (ergocalciferol) and vitamin D3 (cholecalciferol). While both types help to meet the vitamin D requirements of human beings, they differ in a few important ways, such as their food sources. Vitamin D3 is mainly found in animal-sourced foods, such as oily fish including salmon, mackerel, herring etc and cod liver oil. On the other hand, vitamin D2 mainly comes from plant and fungi sources (e.g. mushrooms grown in UV light; mushrooms that are sun dried and yeast exposed to UV light).

Vitamin D is an essential vitamin which helps regulate the amount of calcium and phosphate in the body. These minerals are needed to keep bones, teeth and muscles healthy. Lack of vitamin D can lead to bone diseases such as rickets in children or osteomalacia and osteoporosis in adults. Lack of vitamin D increases the risk of bone fractures in elderly people because it causes fragile bones. Taking too much vitamin D supplements over a long period may cause toxicity including hypercalcemia, kidney stones and soft tissue calcifications including kidneys and blood vessels. In the human body vitamin D is created by the interaction of direct sunlight on the bare skin when outdoors. However, a variety of factors including season, time of day, latitude, skin pigmentation can dramatically influence how much vitamin D is produced in the skin during sun exposure. For example, throughout the year a sufficient amount of vitamin D is produced in the skin upon exposure to sunlight only between about 9 AM and 3 PM local time even when living near the equator. Living above and below 35° latitude results in the sun's angle becoming more oblique and therefore the vitamin D producing rays are absorbed by the ozone layer, thereby markedly reducing the production of vitamin D in the skin during the late fall, winter and early spring months. Modern windows and skin pigmentation are effective sunscreens which absorb solar ultraviolet radiation markedly thereby reducing the skin's ability to produce vitamin D. Because of these variables it has become extremely difficult in modern society to obtain enough vitamin D to satisfy the body's requirement just from sun exposure. This is the reason why it has been estimated that more than 60% of the world's population is vitamin D deficient or insufficient. This in spite of the fact that some foods contain vitamin D naturally and some countries have taken efforts to fortify some foods with vitamin D. This is particularly true for the United States of America, where several foods including milk, dairy products, some juice products and cereals are supplemented with vitamin D (Holick M F; “Vitamin D Deficiency”; N Engl J Med (July 2007) 357:266-281). However, the amount supplemented in this way is relatively little when compared to what is required for children and adults. It was reported in the United States that no child or adult is able to get enough vitamin D from dietary sources. Therefore, there has been an effort to encourage children and adults to take a supplement containing vitamin D to satisfy their body's requirement. Vitamin D containing vitamin supplements are available in health food stores, drugstores and in pharmacies.

Currently recommended dosages of vitamin D are as follows:
for babies up to the age of 1 year 10 to 25 micrograms (1 μg corresponds to 40 International Units) a day are recommended by the US Institute of Medicine and Endocrinology Society Practice Guidelines on vitamin D (Holick M F, Binkley N C, Bischoff-Ferrari H A, Gordon C M, Hanley D A, Heaney R P, Murad M H and Weaver C M; “Evaluation, treatment, and prevention of vitamin D deficiency: an Endocrine Society Clinical Practice Guideline”; J Clin Endocrinol Metab. (December 2011) 96(12): 1911-1930).
for children of 1 year of age and older at least 15 μg and up to 25 μg of vitamin D daily;
adults need at least 15 μg and up to 50 μg of vitamin D a day. This includes people at risk of vitamin D insufficiency or deficiency. It is also recognized that obese adults require at least 2 to 3 times more vitamin D to satisfy the body requirements.

In 2020 a Fact Sheet was issued by the NIH relating to Vitamin D recommendations for health professionals which fact Sheet is currently available under the following link: https://ods.od.nih.gov/factsheets/VitaminD-HealthProfessional/ last assessed Mar. 11, 2021.

Vitamin D is metabolized to 25-hydroxyvitamin D (abbreviated 25(OH)D) which is the major circulating form of vitamin D in the bloodstream. 25(OH)D is measured in the blood by physicians or other health professionals to determine a person's “vitamin D status”, viz. to determine if they are vitamin D deficient, insufficient, sufficient or overdosed (viz. dosed with a toxic dose of vitamin D). Vitamin D is fat-soluble and when ingested is incorporated into chylomicron micelles, which are first released into the lymphatic system before Vitamin D enters the venous blood (Hossein-nezhad A, Holick M F; “Vitamin D for Health: A Global Perspective”; Mayo Clin Proc. (July 2013) 88(7):720-755). 25(OH)D3 is more hydrophilic than vitamin D3 because it has an additional hydroxyl group and therefore is absorbed directly from the gastrointestinal tract and is transported via the hepatic portal vein into the blood system (Jacobs E T, Haussler M R, Alberts D S, Kohler L N, Lance P, Martinez M E, Roe D J, Jurutka P W; “Association between Circulating Vitamin D Metabolites and Fecal Bile Acid Concentrations”; Cancer Prev Res (Phila); (July 2016) 9(7):589-97). Persons with fat malabsorption syndromes who are unable to form adequate amounts of micelles and chylomicrons have a difficult time in absorbing dietary and supplemental vitamin D resulting in vitamin D deficiency (Jacobs E T et al. Cancer Prev Res (Phila); (July 2016); cited above; Margulies S L, Kurian D, Elliott M S, Han Z; “Vitamin D deficiency in patients with intestinal malabsorption syndromes—think in and outside the gut”; J Dig Dis (November 2015) 16(11):617-33). It is therefore suggested that administering 25(OH)D instead of vitamin D may be more successful for improving the vitamin D status, viz. serum 25(OH)D level, in patients with a fat malabsorption syndrome.

Vitamin D insufficiency or deficiency is almost endemic in modern societies (Holick M F; “Vitamin D Deficiency”; N Engl J Med (July 2007) 357:266-281)—for a number of reasons. It accentuates a significant number of health threats, whereby the proof of the respective causal relationships is impeded by the fact that the factors contributing to vitamin D insufficiency or deficiency impact those threats in other ways as well—to differing extents, directly or indirectly, and through mechanisms with or without obvious relation to the individuals' vitamin D status. Nevertheless, there is broad agreement amongst health professionals that an adequate vitamin D status is a very relevant target to be pursued (Holick M F, Binkley N C, Bischoff-Ferrari H A, Gordon C M, Hanley D A, Heaney R P, Murad M H and Weaver C M; “Evaluation, treatment, and prevention of vitamin D deficiency: an Endocrine Society Clinical Practice Guideline”; J Clin Endocrinol Metab. (December 2011) 96(12): 1911-1930). This status is routinely defined in terms of the blood level of 25-hydroxyvitamin D, also referred to as 25(OH)D (viz. 25(OH)D2 and 25(OH)D3), the main metabolite(s) of vitamin D3 and D2 formed in the liver with a long half-life in blood and high affinity to the vitamin D binding protein (DBP). These metabolites are the immediate precursor(s) of the highly active (calcitriol) (1α,25(OH)2-cholecalciferol; or 1α,25(OH)2 vitamin D3; abbreviated as 1,25(OH)2D3) and 1α,25(OH)2-ergocalciferol; or 1α,25(OH)2 vitamin D2; abbreviated as 1,25(OH)2D2, which are jointly abbreviated as 1,25(OH)2D and are responsible for the main effects of vitamin D in the body.

However, not all individuals run the same risk of developing vitamin D insufficiency or deficiency, and the individual risk depends on a number of factors, like the constitution, diet and lifestyle (including sun-exposure). In particular, there is a correlation of the BMI (body mass index) and the risk of developing vitamin D insufficiency and/or deficiency (Wortsman J, Matsuoka L Y, Chen T C, Lu Z and Holick M F; “Decreased bioavailability of vitamin D in obesity”; Am J Clin Nutr (2000) 72:690-693; Ekwaru J P, Zwicker J D, Holick M F, Giovannucci E and Veugelers P J; “The importance of body weight for the dose response relationship of oral vitamin D supplementation and serum 25-hydroxyvitamin D in healthy volunteers”; PLoS One. (November 2014) 9(11):1-11). The higher the BMI, the higher is the risk for developing vitamin D insufficiency and/or deficiency. Thus, it is well-known that obese need significantly higher doses of vitamin D supplementation than non-obese to normalize a low vitamin D status. Therefore, MDs and the medical literature are nowadays advocating the supplementation of significantly increased dosages of vitamin D to obese (2.5 to 3 times more than to non-obese; see Holick M F et al.; JCEM 2011; cited above), and even those high dosages do eventually not suffice to achieve the recommended blood level of 25(OH)D (≥30 ng/mL is the most frequently cited value).

The literature explains the obese subjects' high vitamin D-requirement quite unanimously by the quick and extensive transfer of the non-polar vitamin D from blood to fatty tissues and by their proportionally increased availability of those tissues (Wortsman et al. Am J Clin Nutr (2000); cited above). Thereby this deposition of vitamin D in fatty tissues made evolutionary sense as it provided a reserve to be exploited in times of reduced sunlight (in winter!) leading to a significantly reduced endogenous vitamin D3 formation. But it has lost a lot of its meaning in modern civilizations with their vitamin D supplementation and reinforcement of diet-components consumed throughout the year. In other words: The trapping of vitamin D in fatty tissues, in particular in over-weight and obese, is now rather being recognized as a problem in view of the target to reach healthy blood levels of 25(OH)D (see: Cesaro R, Falchetti A, Attanasio R, Tabacco G, Naciu A M and Palermo A; “Hypovitaminosis D: Is it time to consider the use of calcifediol?”; Nutrients (May 2019) 11(5):1016-1035).

Studies of the vitamin D status in patients with anorexia nervosa have shown that the respective patients have a low prevalence of vitamin D deficiency. It has been postulated that these malnourished patients, who possess minimal body fat, have a decreased metabolic clearance and decreased uptake of vitamin D into the adipose tissue. This is supported by a clinical study (DiVasta A D, Feldmann H A, Brown J N, Giancaterino C, Holick M F, and Gordon C M; “Bioavailability of vitamin D in malnourished adolescents with anorexia nervosa”; J Clin Endocrinol and Metab (August 2011) 96(8):2575-2580) which has shown that patients with anorexia nervosa have relatively normal concentrations of 25(OH)D compared with normative standards or healthy control subjects. However, these anorexic patients also were found to have at baseline circulating blood levels of vitamin D to be more than 300% higher compared to healthy normal weight control subjects. Thus, undesired vitamin D uptake from lymph into fat tissue may be comparatively much more relevant to obese persons. In normal or underweight persons, the vitamin D in the circulation enters the body fat. Anorexic patients have so little body fat that the vitamin D remains in the bloodstream. In persons of normal weight vitamin D still enters the body fat but is then readily released again into the circulation, where it is transformed to 25(OH)D in the liver. In obese patients the vitamin D is sequestered viz. diluted in the body fat and therefore very little vitamin D is released back into the circulation. This results in less vitamin D in the bloodstream, and therefore less vitamin D can be converted to 25(OH)D causing that obese persons have lower blood levels of 25(OH)D and therefore an increased risk of being vitamin D insufficient or deficient compared to a normal weight and overweight adult.

In WO 2009/10132 it is disclosed that plasma 25(OH)D3 increases synergistically when a person is administered a combination of vitamin D (cholecalciferol and/or ergocalciferol) and 25-hydroxyvitamin D3 (calcifediol) and that the said combination synergistically regulates (either up-regulates or down-regulates) a synergistic number of vitamin D responsive genes, including a high number of genes which are not responsive to the presence of either vitamin D or 25(OH)D3 alone. In particular, the said combination is recommended for the treatment of vitamin D deficiency in elderly people.

In WO 2009/047644 it is disclosed that co-administration of 25-hydroxyvitamin D3 with 25-hydroxyvitamin D2 can more effectively elevate serum levels of 25-hydroxyvitamin D without causing toxicity than administration of either alone. The combination is said to be useful for treating any subject in need of vitamin D supplementation, either prophylactically to prevent vitamin D insufficiency or deficiency, or therapeutically to replete low serum vitamin 25(OH)D levels in a wide variety of disease states, including e.g. subjects with obesity (vitamin D deposited in body fat tissues is less bioavailable). The ratio of the 25(OH)D3 supplement and the 25(OH)D2 supplement is disclosed to be in the range of 100 to 1 and 1 to 20 and is preferably at least 1 to 1, 1.5 to 1 or 2 to 1.

In US 2019/0298744 A1 it is disclosed that calcifediol (25(OH)D3) may be used to treat bariatric surgery patients. Also disclosed is treating or preventing one or more complications of vitamin D insufficiency or deficiency associated with obesity and bariatric surgery. The patent's focus is on patients under medical treatment for obesity, not on healthy individuals. Moreover, the use of combinations as disclosed in the present patent application is not disclosed in US 2019/0298744 A1. However, it considers in essence the whole range of conceivable vitamin D supplementation options as tolerable in parallel to the treatment.

In a recently published review (Cesaro R, Falchetti A, Attanasio R, Tabacco G, Naciu A M and Palermo A; “Hypovitaminosis D: Is it time to consider the use of calcifediol?”; Nutrients (May 2019) 11(5):1016-1035) it is disclosed that oral calcifediol (25(OH)D3) is about three- to five-time more powerful than oral cholecalciferol (vitamin D3), and that it has a higher rate of intestinal absorption. Based on this it is suggested that calcifediol can be particularly useful in treating diseases associated with decreased intestinal absorption, as well as obesity (given its lower trapping in adipose tissue).

A significant number of patients with intestinal malabsorption syndromes, including celiac disease (CD), cystic fibrosis (CF), short bowel syndrome, inflammatory bowel disease (IBD), hepatic dysfunction, gastric bypass surgery and intestinal lymphangiectasia among other disorders are at a high risk for vitamin D deficiency because of their inability to efficiently absorb vitamin D in their gastrointestinal tract (Jacobs E T et al. Cancer Prev Res (Phila); (July 2016); cited above; Margulies S L et al.; cited above). A vitamin D absorption test has been developed that has been helpful in determining the efficiency of vitamin D absorption in patients with intestinal fat malabsorption syndromes. Patients and normal healthy adults took a single oral dose of 1.25 mg (1250 μg or 50′000 IU) of vitamin D, and blood levels of vitamin D were then determined. The serum vitamin D concentration rose from a baseline of less than 5 ng/mL to a peak of over 50 ng/mL by 12 h, gradually falling to baseline levels by 3 days. In five of the seven patients with intestinal fat malabsorption, oral administration of 50,000 IU vitamin D2 did not raise serum vitamin D concentrations above 10 ng/mL. Two patients with severe inflammatory bowel disease had a normal absorption pattern, however. These findings suggest that an oral vitamin D absorption test may be of value for determination of persons at risk for development of vitamin D insufficiency or deficiency. The results give raise to questions about the efficacy of oral vitamin D preparations in patients with intestinal fat malabsorption (Lo C W, Paris P W, Clemens T L, Nolan J, and Holick M F; “Vitamin D absorption in healthy subjects and in patients with intestinal malabsorption syndromes”; Am. J. of Clin. Nutr. (October 1985) 42:644-649; and Farraye F A, Nimitphong H, Stucchi A, Dendrinos K, Boulanger A B, Vijjeswarapu A, Tannenbaum A, Biancuzzo R, Chen T C and Holick M F; “Use of a novel vitamin D bioavailability test demonstrates that vitamin D absorption is decreased in patients with quiescent Crohn's Disease”; Inflamm Bowel Dis. (October 2011) 17(10): 2116-2121).

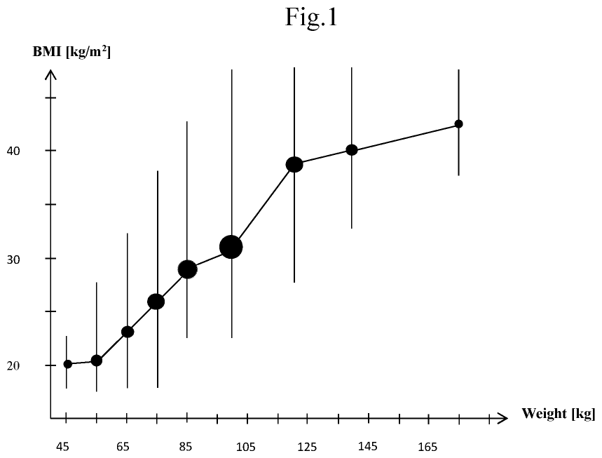

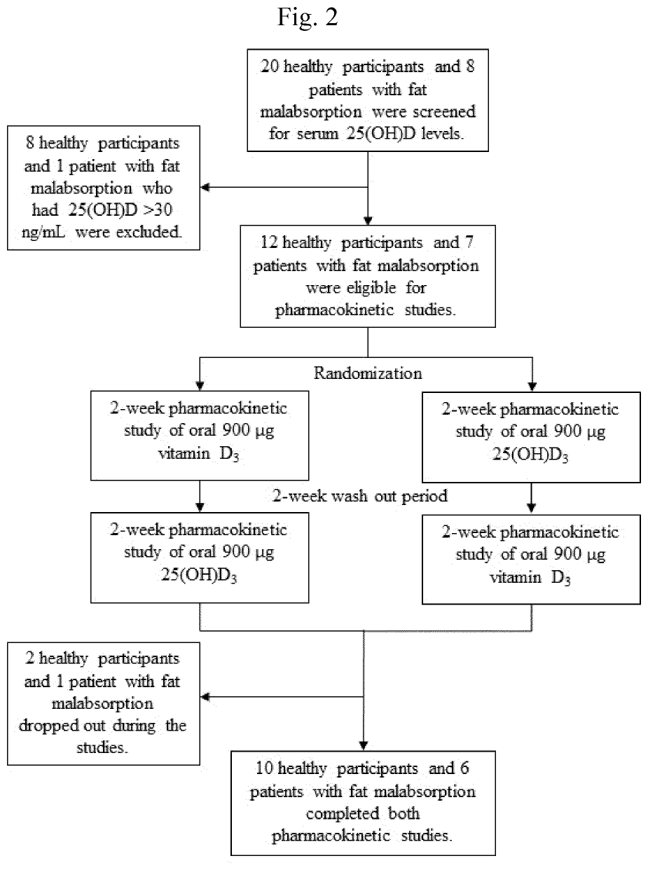

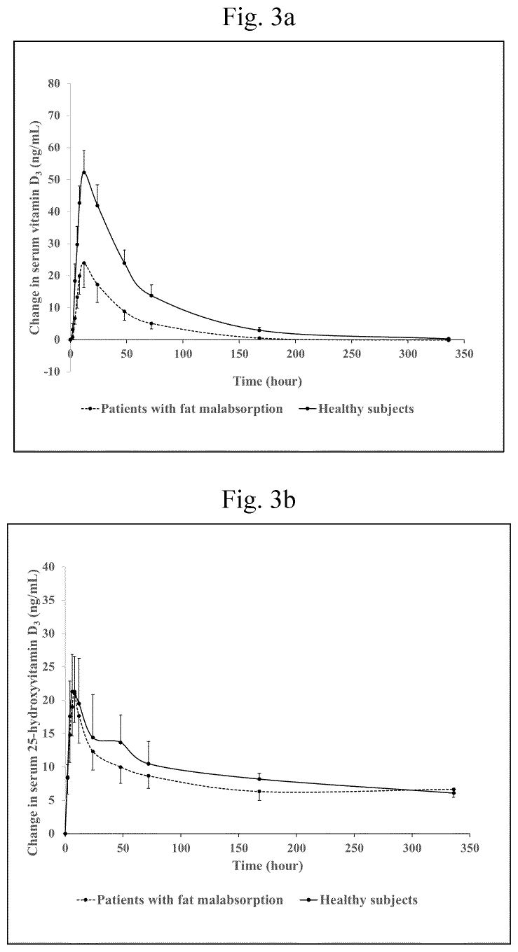

Recently the FDA approved a slow release 25(OH)D3 formulation (see Sprague S M, Strugnell S A and Bishop C W (2017); “Extended-release calcifediol for secondary hyperparathyroidism in stage 3-4 chronic kidney disease”; Expert Review of Endocrinology & Metabolism (2017) 12(5) 289-301; and Sprague S M, et al.; “Modified-release calcifediol effectively controls secondary hyperparathyroidism associated with vitamin D insufficiency in chronic kidney disease”; Am J Nephrol. (January 2015) 40:535-545). Pharmacokinetic studies with an oral single dose of 900 μg of 25(OH)D3 (FIG. 3 of Sprague et al. 2017; cited above) and an oral single dose of 1250 μg of vitamin D (FIGS. 1 and 2 of Lo et al.; Am. J. of Clin. Nutr. (October 1985); cited above and Farraye F A et al; Inflamm Bowel Dis. (October 2011); cited above), respectively did not show any untoward toxicity.

The publication of van Groningen L, Opdenoord S, van Sorge A, Telting D, Glesen A and de Boer H; “Cholecalciferol loading dose guideline for vitamin D-deficient adults”; Europ. J. of Endocrinology (2010) 162: 805-811 recommends another approach, viz. administering a total “loading dose” over many weeks and is based on the following formula: Dose in I.U. of vitamin D=40×(75 [nmol/L]−nmol/L [initial blood level 25(OH)D3])×body weight [kg].

This defines a dose of, e.g., 200 000 I.U. for an individual weighing 100 kg and having a 25(OH)D blood level of 10 ng/mL. Thereby the publication by van Groningen et al. explicitly excludes obese people, defining obesity as body weight >120 kg (!), and underlines that the required dosage depends on the body weight (BW) which appears in the formula, but not the BMI—that no correlation with the BMI was found.

Jetter et al. have reported, inter alia, levels of 25(OH)D3 achieved in vitamin D deficient postmenopausal women after single p.o. bolus doses of 140 μg of vitamin D3, of 140 μg of 25(OH)D3, and of 140 μg of vitamin D3 plus 140 μg of 25(OH)D3, whereby Cₘₐₓ reached increases over baseline of 5.4, 24.7, and 27.2 ng/mL, respectively, showing that there is no statistically significant impact of the addition of vitamin D3 upon the effect of 25(OH)D3 (confirmed by the AUCs) (Jetter A., Egli A., Dawson-Hughes B., Staehelin H. B., Stoecklin, E. Goessi, R., Henschowski J., Bischoff-Ferrari H. A.; «Pharmacokinetics of oral vitamin D3 and calcifediol»; Bone (2014) 59: 14-19. It is emphasized that Jetter et. al. did their study in vitamin D deficient postmenopausal women and did not study the situation in in respect of obesity. In addition, the dosing used by Jetter et al. is different from the one disclosed in the present patent application.

### DETAILED DESCRIPTION OF THE INVENTION

As outlined above a vitamin D deficiency and/or insufficiency, viz. a low blood level of 25(OH)D, is up to now mainly treated by providing vitamin D in form of vitamin D supplements. This works well in normal subjects having a normal uptake of vitamin D. However, there are persons with a vitamin D insufficiency or deficiency who are “true” malabsorbers, viz. the uptake of vitamin D per se into blood in these persons is comperatively poor. Malabsorbers are e.g. people after bariatic surgery and persons with inflammatory bowel diseases. However, this list is not meant to be limiting and other individuals may also be classified as malabsorbers. Healthy obese persons may not show malabsorption. In medical practice vitamin D deficiencies are often treated by increasing the vitamin D dosage in order to reach normal blood levels. It has been recognized that the vitamin D supplemented to obese persons is predominantly stored in their fat. Therefore, trying to normalize the vitamin D status in obese persons by administering vitamin D2 or vitamin D3 is per se very inefficient. Therefore, novel methods and compositions for managing obesity-related vitamin D insufficiency and/or definciency are desirable.

Of further concern is the still largely unexplored fate and role of high dosages of vitamin D and its metabolites in fatty tissues—including the impact of those substances upon the homeostasis of the fatty tissue itself. According to the literature available to the skilled person, the mass of adipocytes, containing to a very large extent in their lipid droplets, is responsible for the comparatively very rapid disappearance of vitamin D from blood observed when administering the lipophilic vitamin D to obese individuals with their comparatively strongly increased mass of adipocytes (cp. A. Hengist et al., Nutrition Bulletin 2019, “Mobilising vitamin D from adipose tissue: The potential impact of exercise” available under the link https://onlinelibrary.wiley.com/doi/full/10.1111/nbu.12369, as accessed on Mar. 11, 2021). Susanne Prattes et al. (Journal of Cell Science 113, 2977-2989 (2000) https://jcs.biologists.org/content/joces/113/17/2977.full.pdf) report that cholesterol which is structurally related to vitamin D, is not ending up in the lipid droplets, in spite of its lipophilicity. Instead, it is distributed to their surface and to the adipocytes' cellular membrane. The present inventors have found that surprisingly the same may likely hold for vitamin D. This interpretation is supported by the results shown in the Examples further below, in which the blood levels of vitamin D per se after its oral administration to obese and normal BMI subjects are measured. It is against the prejudice and misconception in the field that significant amounts of this vitamin, if administered to obese individuals, go into the lipid droplets and become physiologically inaccessible for certain, perhaps extended, time. Rather they stay accessible and impact the physiology of the adipocytes themselves. While it cannot be excluded at this point that some administered vitamin D may after certain time migrate to the inner layers of lipid droplets, it is assumed that this diffusion is not rapid enough to significantly influence the initial distribution after uptake into blood. Therefore, it is to be understood that the respective surface, rather than the mass, determines the uptake of vitamin D into fatty tissues.

The effects of vitamin D and its metabolites upon the adipocytes themselves shall be discussed before further addressing the pharmacokinetics. This discussion raised significant recent interest and resulted in several publications, which project a conflicting picture, for which several reasons exist. One is that fatty tissues differ in type (brown fatty tissue versus white fatty tissue), their anatomical localization, and their state of maturation. Another point responsible for the divergent answers to the question whether the vitamin D metabolites, in particular 1,25(OH)2D, promote or inhibit the growth of adipose tissues and promote or inhibit apoptosis of adipocytes, respectively, may have been that those metabolites' inefficient uptake into adipocytes depends on the experimental conditions studied and thus varies in different experimental setups. Therefore, studies employing the metabolites may not really reflect the effects of vitamin D per se, which is easily taken up into adipocytes and known to be metabolized there.

Accordingly, the present inventors have found that surprisingly there must be optimal conditions of supplementation of vitamin D to adipocytes in particular with respect to the effect of said supplementation on vitamin D homeostasis. The present inventors have further found that it must surprisingly be close to a reasonable optimum to supplement loads of vitamin D to adipocytes of obese individuals, which result in surface densities comparable to the ones found to be ideal for the adipocytes of non-obese normal BMI persons.

These findings hold true for healthy and unhealthy obese individuals. Still, the optimum dosages to be administered to those two subpopulations are different. Obesity as it progresses is becoming more and more unhealthy, eventually becoming a disease, especially when it is further characterized by signs of inflammation, including large numbers of macrophages in the lymphatic vessels' boundaries and adipose tissues. These macrophages metabolize vitamin D, thus reducing its uptake into adipocytes. Therefore, the relative dosages of vitamin D to be given to unhealthily obese persons (who may preferably be regarded as patients) have to be higher than the ones for healthy obese. While it is true that there is a basic correlation of increasing BMI with increasing “unhealthiness” (corresponding to an increasing inflammatory character) of obesity, there are individuals who do not have a tremendously high BMI, but do already have a strong inflammatory character of their obesity. One point which is undoubtedly playing a role is thereby the duration (for how many years has a person maintained a high BMI).

The supplemented vitamin D plays a dual role. In one role, it acts as precursor of 25(OH)D which defines the vitamin D status, is formed in the liver, is with high affinity complexed to DBP, has correspondingly a long half-life in blood, may be transitorily deposited in different types of tissues, and can eventually be, if not degraded, transformed to 1,25(OH)2D. Whereby 1,25(OH)2D is the highly active final metabolite which plays a role in many types of cells. The second role vitamin D plays is upon being deposited in adipocytes, getting metabolized in adipocites and influencing the adipocytes' homeostasis (Nimitphong H, Guo W, Holick M F, Fried S K, Lee M J. Vitamin D Inhibits Adipokine Production and Inflammatory Signaling Through the Vitamin D Receptor in Human Adipocytes. Obesity (Silver Spring). 2021 March; 29(3):562-568. doi: 10.1002/oby.23109. PMID: 33624437).

Vitamin D can be released again eventually. In non-obese normal BMI individuals the supplemented vitamin D plays those two roles in a balanced way, provided the dosages aren't heavily exaggerated. In overweight and obese individuals and in individuals with a tendency to get obese, however, it may be impossible to supplement vitamin D in amounts/dosage leading to an adequate (blood level-based) vitamin D status without overdosing adipose tissues and negatively influencing their homeostasis, e.g. by further promoting obesity. This problem can be solved by the approach described for the first time in the present patent application, namely that limited doses of vitamin D tailored to optimally contribute to the homeostasis of adipose tissues, are to be combined with an amount of 25(OH)D sufficient to reach a healthy vitamin D status.

Vitamin D3 and vitamin D2 are converted in the liver to 25(OH)D. These vitamin D metabolites, which have a long T_{1/2} in blood, represent actually the major circulating form in the blood plasma. In fact, the level of 25(OH)D is regularly used to determine the “vitamin D status” of a person. 25(OH)D is the immediate precursor of the highly active calcitriol (1α,25(OH)2-cholecalciferol; or 1α,25(OH)2 vitamin D3; or abbreviated 1,25(OH)2D3) and 1α,25(OH)2-ergocalciferol; or 1α,25(OH)2 vitamin D2; or abbreviated 1,25(OH)2D2 which are responsible for the main effects of vitamin D in the body.

As noted above vitamin D is to a good extent taken up via the lymphatic pathway. On the other hand, 25(OH)D is more polar and therefore to a much larger extent taken up directly into the blood system via the portal vein. It has been observed that there are differences between the lymphatic fluxes in obese persons compared to persons of average, viz. normal weight. Moreover, it has been found that the transfer into fatty tissues (adipocytes) of (mainly non-polar) substances like vitamin D is comparatively increased in obese persons, once these substances arrive in the blood circulation. Persons of normal weight (viz. non-obese persons) have a faster lymphatic flux, which implies that the substances taken up into the lymph are much more rapidly ending up in the bloodstream, e.g. via the subclavian vein. In other words, vitamin D being fat soluble has a propensity to be incorporated into the body fat. In persons with a lot of body fat the vitamin D has a greater opportunity to enter the body fat pool and a more difficult time exiting from there. What has, however, not been considered up to now is that non-polar substances like vitamin D may, particularly in obese persons, pass in part directly from the lymph to fatty tissues (adipocytes).

The vitamin D status of a person is to a certain extent dependent on its lifestyle. A person exposing the skin to sunlight and/or ingesting food that naturally contains or is fortified with vitamin D will most likely achieve enough vitamin D and 25(OH)D levels without much ado. This does not apply to persons who are obese or have a tendency to get obese because the ingested vitamin D is diluted and trapped in the fatty tissues and neither is getting into nor is available long enough in the bloodstream, respectively, to travel to the liver for its transformation to 25(OH)D. However, 25(OH)D taken up by normal weight and obese persons after ingestion is deposited into the bloodstream directly from the small intestine and remains in the blood. As a result, it is available to be transported to the kidneys to be activated. This led to the suggestion to just supplement 25(OH)D3 (or eventually 25(OH)D2) in obese persons instead of vitamin D alone. However, an optimum “vitamin D-status” in terms of adequate levels of vitamin D as well as 25(OH)D in the organism's different compartments may not be reached in that way.

The present invention was made in view of the state of the art as described above. The objective technical problem of the present invention is to provide improved vitamin D supplementation methods and corresponding vitamin D supplements. Particularly desirable are improved methods and/or vitamin D supplements, viz. a medicament, a nutraceutical or a food additive for use in the treatment of and/or prophylaxis of vitamin D insufficiencies or deficiencies, particularly in obese persons, persons having the tendency to get obese and in persons having a history of malabsorption of vitamin D, preferably in obese persons, and in persons having the tendency to get obese, more preferably in obese persons.

The problem is solved by the embodiments described herein and as characterized in the appended claims.

In accordance with the present invention it has been found that the supplementation of vitamin D and 25(OH)D in a ratio between 1 to 5 or 5 to 1, or preferably between 1:2 to 2:1, more preferably in a ratio selected from the group of 1 to 1, 1 to 1.5, 1 to 2, 1.5 to 1, is appropriate in order not to overload the persons' fatty tissue and still normalize the 25(OH)D level in the blood. As referred to herein, a ratio selected from the group of 1 to 1, 1 to 1.5, 1 to 2, 1.5 to 1 is to be understood as a ratio selected from the group of 1:1, 1:1.5, 1:2, 1.5:1.

Furthermore, in accordance with the present invention it is proposed to use either 25(OH)D3 or 25(OH)D2 or a combination of 25(OH)D3 and 25(OH)D2 provided the person has a sufficient level of endogeneous vitamin D2 and/or vitamin D3 in the broadest sense (cp. the above remarks on formation and/or uptake as well as blood levels and endogenous storage); or a combination of 25(OH)D3 and/or 25(OH)D2 and vitamin D2, or a combination of 25(OH)D3 and/or 25(OH)D2 and vitamin D3; or a combination of 25(OH)D3 and/or 25(OH)D2 and a combination of vitamin D2 and vitamin D3 in order to establish sufficient vitamin D and 25(OH)D levels in such persons. In the latter cases preferrably the ratio of the administered vitamin D3 and/or vitamin D2 to the administered 25-hydroxy form of the vitamin D (preferably understood as corresponding 25-hydroxy form) is in the range between 1 to 5 and 5 to 1, between 1 to 4 and 4 to 1 or between 1 to 3 or 3 to 1, preferentially between 1 to 2 and 2 to 1, or in a ratio of 1 to 1, or in a ratio selected from the group of 1 to 1, 1 to 1.5, 1 to 2, and 1.5 to 1. The preferred range may depend on the body mass index (BMI) and/or the lifestyle (including sun-exposure) of the person. It is further noted that preferably the dosage depends on the persons's level of vitamin D insufficiency and/or deficiency, respectively, wherein the level of vitamin D insufficiency or deficiency is statistically dependent on the BMI. Furthermore, preferably within the scope of the present invention, analytical determination of the vitamin D status should be considered when the optimal dosage for the individual in question or the patient, respectively is determined. It is further noted that analytical determination of the vitamin D status becomes more important in the cases of higher BMI (i.e., analytical determination of the vitamin D status becomes more important the higher the BMI is).

One object of the present invention is therefore providing a vitamin D supplement, viz. a medicament, a nutraceutical or a food additive which comprises either 25(OH)D3 or 25(OH)D2, or a combination of 25(OH)D3 and 25(OH)D2; or (i) a combination of 25(OH)D3 and/or 25(OH)D2 and vitamin D2; or (ii) a combination of 25(OH)D3 and/or 25(OH)D2 and vitamin D3; or (iii) a combination of 25(OH)D3 and/or 25(OH)D2 and a combination of vitamin D2 and vitamin D3, for use in the treatment of and/or prophylaxis of vitamin D insufficiency or deficiency in obese persons, overweight persons having the tendency to get obese or persons having a history of malabsorption of vitamin D or persons with a genetic disposition to develop obesity, preferably in obese persons, and in overweight persons having the tendency to get obese, more preferably in obese persons.

In one aspect the said use of a vitamin D supplement, viz. a medicament, a nutraceutical or a food additive which comprises either 25(OH)D3 or 25(OH)D2, or a combination of 25(OH)D3 and 25(OH)D2 is dependent on the level of endogeneous vitamin D2 and/or vitamin D3 per se of the person treated and is preferably dependent on the amounts of vitamin D2 ingested and/or the amounts of vitamin D3 ingested and/or generated, respectively, by the treated person.

Another object of the invention is providing a vitamin D supplement, viz. a medicament, a nutraceutical or a food additive which comprises a combination of 25(OH)D3 and 25(OH)D2; or (i) a combination of 25(OH)D3 and/or 25(OH)D2 and vitamin D2; or (ii) a combination of 25(OH)D3 and/or 25(OH)D2 and vitamin D3; or (iii) a combination of 25(OH)D3 and/or 25(OH)D2 and a combination of vitamin D2 and vitamin D3 for use in the treatment of and/or prophylaxis of vitamin D insufficiency or deficiency in obese persons, overweight persons having the tendency to get obese or persons having a history of malabsorption of vitamin D or persons with a genetic disposition to develop obesity, preferably in obese persons, and in overweight persons having the tendency to get obese, more preferably in obese persons.

In one aspect the said use of a vitamin D supplement, viz. a medicament, a nutraceutical or a food additive which comprises either 25(OH)D3 or 25(OH)D2, or a combination of 25(OH)D3 and 25(OH)D2 is dependent on the level of endogeneous vitamin D2 and/or vitamin D3 per se of the person treated, preferably dependent on the amounts of vitamin D2 ingested and/or the amounts of vitamin D3 ingested and/or generated, respectively, by the treated person.

Another object of the present invention is providing a vitamin D supplement, viz. a medicament, a nutraceutical or a food additive which comprises (i) a combination of 25(OH)D3 and/or 25(OH)D2 and vitamin D2; or (ii) a combination of 25(OH)D3 and/or 25(OH)D2 and vitamin D3; or (iii) a combination of 25(OH)D3 and/or 25(OH)D2 and a combination of vitamin D2 and vitamin D3, for use in the treatment of and/or prophylaxis of vitamin D insufficiency or deficiency in obese persons, overweight persons having the tendency to get obese or persons having a history of malabsorption of vitamin D or persons with a genetic disposition to develop obesity, preferably in obese persons, and in overweight persons having the tendency to get obese, more preferably in obese persons.

In another aspect each of the said uses is characterized in that the ratio of the administered amount of vitamin D2, vitamin D3, or their combined amounts to the administered 25-hydroxy form(s) of the vitamin D is in the range between 1 to 5 and 5 to 1, between 1 to 4 and 4 to 1 or between 1 to 3 or 3 to 1, preferentially between 1 to 2 and 2 to 1, or in a ratio selected from the group of 1 to 1, 1 to 1.5, 1 to 2, 1.5 to 1.

In another aspect each of the said uses is characterized in that the ratio of the administered amount of vitamin D2, vitamin D3, or their combined amounts to the administered 25-hydroxy form(s) of the vitamin D is in the range between 1 to 3 or 3 to 1, between 1 to 2 and 2 to 1, or preferentially in a ratio selected from the group of 1 to 1, 1 to 1.5, 1 to 2, 1.5 to 1.

In one aspect each of the said uses is characterized in that the ratio of the administered amount of vitamin D2, vitamin D3, or their combined amounts to the administered 25-hydroxy form(s) of the vitamin D is in the range between 1 to 5 and 5 to 1, preferentially between 1 to 2 and 2 to 1, or in a ratio selected from the group of 1 to 1, 1 to 1.5, 1 to 2, 1.5 to 1.

In another aspect each of the said uses is characterized in that the ratio of the administered amount of vitamin D2, vitamin D3, or their combined amounts to the administered 25-hydroxy form(s) of the vitamin D is optimized based on the body mass index (BMI) of the person and/or the lifestyle of the person.

In another aspect each of the said uses is characterized in that the ratio of the administered amount of vitamin D2, vitamin D3, or their combined amounts to the administered 25-hydroxy form(s) of the vitamin D is optimized based on eventual condition of a person resulting in vitamin D malabsorption and/or on the lifestyle-dependent endogenous formation of vitamin D3 and/or on the diet-dependent intake of vitamin D. Said condition may determine whether the 25-hydroxy form(s) of the vitamin D are administered to the person either alone or in combination with vitamin D2 and/or vitamin D3.

In another aspect each of the said uses is characterized in that the 25-hydroxy form(s) of the vitamin D are administered to an obese person either alone or in combination with modest amounts of vitamin D2 and/or vitamin D3 in order to avoid the risk of counterproductive or negative effects due to accumulation of comparatively higher amounts of vitamin D alone. In this connection modest amount(s) is defined as the amount of vitamin D supplement appropriate for a person of normal weight, viz. a person with a body mass index of about 25. It is noted that supplementing elevated doses of vitamin D to obese persons should and can be avoided, if combining it with relevant doses of 25(OH)D.

In another aspect each of the said uses is characterized by administering to an obese person at least approximately the amounts of vitamin D which are sufficient to establish or maintain an adequate vitamin D status in non-obese subjects without genetic disposition to develop obesity and in addition supplementing the required amount of 25(OH)D in order to reach a sufficient vitamin D status.

In another aspect each of the said uses is characterized by administering at least approximately the amounts of vitamin D which are sufficient to establish or maintain an adequate vitamin D status in non-obese subjects without genetic disposition to develop obesity and in addition administering the required amount of 25(OH)D2 or 25(OH)3, or a combination of 25(OH)D2 and 25(OH)D3.

In another aspect the vitamin D supplement in accordance with the present invention comprises vitamin D and 25(OH)D in a daily dose selected from the group as follows:
(i) For slightly obese individuals of unknown or unreliably known level of 25(OH)D in blood, respectively, who have a normal lifestyle with respect to UV-light exposure and daily uptake of vitamin D, the dose comprises vitamin D and 25(OH)D in a ratio of 1 to 1 and in amounts at the lower end of the range of 5 to 7.5 μg of each of the two products;
(ii) For slightly obese individuals of unknown or unreliably known level of 25(OH)D in blood, respectively, who have an unfavorable lifestyle with respect to UV-light exposure and daily uptake of vitamin D, the dose comprises vitamin D and 25(OH)D in a ratio of 1 to 1 and in amounts at the higher end of the range of 5 to 7.5 μg of each of the two products;
(iii) For slightly obese individuals of reliably known and not much too low level of 25(OH)D in blood (>20 to 30 ng/mL) the dose comprises vitamin D and 25(OH)D in a ratio of 1 to 1 and in amounts in the range of 5 to 7.5 μg of each of the two products;
(iv) For slightly obese individuals of reliably known and clearly low level of 25(OH)D in blood (>10 to 20 ng/mL) the dose comprises vitamin D and 25(OH)D in a ratio of 1 to 1.5, keeping the dose of vitamin D at 5 to 7.5 μg and raising the dose of 25(OH)D to 7.5 to 11 μg;
(v) For slightly obese individuals of reliably known and very low level of 25(OH)D in blood (≤10 ng/mL) the dose comprises vitamin D and 25(OH)D in a ratio of 1 to 2, keeping the dose of vitamin D at 5 to 7.5 μg and raising the dose of 25(OH)D to 10 to 15 μg;
(vi) For clearly obese individuals of unknown or unreliably known level of 25(OH)D in the blood, respectively, who have a normal lifestyle with respect to UV-light exposure and daily uptake of vitamin D, the dose comprises vitamin D and 25(OH)D in a ratio of 1 to 1 and in amounts at the lower end of the range of 7.5 to 11 μg of each of the two products;
(vii) For clearly obese individuals of unknown or unreliably known level of 25(OH)D in the blood, respectively, who have an unfavorable lifestyle with respect to UV-light exposure and daily uptake of vitamin D, the dose comprises vitamin D and 25(OH)D in a ratio of 1 to 1 and in amounts at the higher end of the range of 7.5 to 11 μg of each of the two products;
(viii) For clearly obese individuals of reliably known and not much too low level of 25(OH)D in blood (>20 to 30 ng/mL) the dose comprises vitamin D and 25(OH)D in a ratio of 1.5 to 1, keeping the dose of vitamin D at 7.5 to 11 μg, which results in a dose of 25(OH)D of 5 to 7.5 μg;
(ix) For clearly obese individuals of reliably known and clearly low level of 25(OH)D in blood (>10 to 20 ng/mL) the dose comprises vitamin D and 25(OH)D in a ratio of 1 to 1 and in amounts of 7.5 to 11 μg of each of the two products;
(x) For clearly obese individuals of reliably known and very low level of 25(OH)D in blood (≤10 ng/mL) the dose comprises vitamin D and 25(OH)D in a ratio of 1 to 1.5, keeping the dose of vitamin D at 7.5 to 11 μg, which results in a dose of 25(OH)D of 11 to 17 μg;
(xi) For extremely obese individuals of unknown or unreliably known level of 25(OH)D in blood, respectively, who have a normal lifestyle with respect to UV-light exposure and daily uptake of vitamin D, the dose comprises vitamin D and 25(OH)D in a ratio of 1 to 1 and in amounts at the lower end of the range of 11 to 15 μg of each of the two products;
(xii) For extremely obese individuals of unknown or unreliably known level of 25(OH)D in blood, respectively, who have an unfavorable lifestyle with respect to UV-light exposure and daily uptake of vitamin D, the dose comprises vitamin D and 25(OH)D in a ratio of 1 to 1 and in amounts at the higher end of the range of 11 to 15 μg of each of the two products;
(xiii) For extremely obese individuals of reliably known and not much too low level of 25(OH)D in blood (>20 to 30 ng/mL) the dose comprises vitamin D and 25(OH)D in a ratio of 1.5 to 1, keeping the dose of vitamin D at 11 to 15 μg, which results in a dose of 25(OH)D of 7.5 to 10 μg;
(xiv) For extremely obese individuals of reliably known and clearly low level of 25(OH)D in blood (>10 to 20 ng/mL) the dose comprises vitamin D and 25(OH)D in a ratio of 1 to 1 and in amounts of 11 to 15 μg of each of the two products;
(xv) For extremely obese individuals of reliably known and very low level of 25(OH)D in blood (≤10 ng/mL) the dose comprises vitamin D and 25(OH)D in a ratio of 1 to 1.5, keeping the dose of vitamin D at 11 to 15 μg, which results in a dose of 25(OH)D of 17 to 22.5 μg.

In a preferred embodiment the vitamin D supplement comprising vitamin D and 25(OH)D in a dose selected from the groups specified above comprises vitamin D3 and 25(OH)D3, respectively.

In another aspect each of the said uses is characterized by administering to a person with a deficient or insufficient 25(OH)D level and having a BMI of between 30 and 36 between 5.0 μg and 7.5 μg vitamin D, preferably vitamin D3, plus between 5.0 μg and 7.5 μg 25(OH)D, preferably 25(OH)D3.

In another aspect each of the said uses is characterized by administering to a person with a deficient or insufficient 25(OH)D level and having a BMI of between 37 and 43 between 7.5 μg and 11.25 μg of vitamin D, preferably vitamin D3, plus between 7.5 μg and 11.25 μg of 25(OH)D, preferably 25(OH)D3. For the sake of clarity, it is noted that 1 μg of vitamin D corresponds to 40 International Units (IU).

In another aspect each of the said uses is characterized in that the treatment of and/or prophylaxis is for reducing the chance that the person develops a disease which has a relation to obesity such as diabetes and cardiovascular disease.

Another object of the invention is a method of treating a vitamin D insufficiency or deficiency in obese persons, persons having the tendency to get obese and in persons having a history of malabsorption of vitamin D or persons with a genetic disposition to develop obesity, preferably in obese persons, and in persons having the tendency to get obese, more preferably in obese persons, with a vitamin D supplement, viz. a medicament, a nutraceutical or a food additive comprising either 25(OH)D3 or 25(OH)D2, or a combination of 25(OH)D3 and 25(OH)D2; or (i) a combination of 25(OH)D3 and/or 25(OH)D2 and vitamin D2; or (ii) a combination of 25(OH)D3 and/or 25(OH)D2 and vitamin D3; or (iii) a combination of 25(OH)D3 and/or 25(OH)D2 and a combination of vitamin D2 and vitamin D3.

In one aspect the said method of treating a vitamin D insufficiency or deficiency in obese persons, persons having the tendency to get obese and in persons having a history of malabsorption of vitamin D or persons with a genetic disposition to develop obesity, preferably in obese persons, and in persons having the tendency to get obese, more preferably in obese persons, with a vitamin D supplement, viz. a medicament, a nutraceutical or a food additive comprising (i) a combination of 25(OH)D3 and/or 25(OH)D2 and vitamin D2; or (ii) a combination of 25(OH)D3 and/or 25(OH)D2 and vitamin D3; or (iii) a combination of 25(OH)D3 and/or 25(OH)D2 and a combination of vitamin D2 and vitamin D3.

In one aspect the said method is characterized in that the amount of vitamin D supplement, viz. a medicament, a nutraceutical or a food additive which comprises either 25(OH)D3 or 25(OH)D2, or a combination of 25(OH)D3 and 25(OH)D2 is dependent on the level of endogeneous vitamin D2 and/or vitamin D3 per se of the person treated, preferably dependent on the amounts of vitamin D2 ingested and/or the amounts of vitamin D3 ingested and/or generated, respectively, by the treated persons.

Another object of the invention is a method of treating a vitamin D insufficiency or deficiency in obese persons, persons having the tendency to get obese and in persons having a history of malabsorption of vitamin D or persons with a genetic disposition to develop obesity, preferably in obese persons, and in persons having the tendency to get obese, more preferably in obese persons, with a vitamin D supplement, viz. a medicament, a nutraceutical or a food additive comprising a combination of 25(OH)D3 and 25(OH)D2; or (i) a combination of 25(OH)D3 and/or 25(OH)D2 and vitamin D2; or (ii) a combination of 25(OH)D3 and/or 25(OH)D2 and vitamin D3; or (iii) a combination of 25(OH)D3 and/or 25(OH)D2 and a combination of vitamin D2 and vitamin D3.

In one aspect the said method is characterized in that the amount of vitamin D supplement, viz. a medicament, a nutraceutical or a food additive which comprises either 25(OH)D3 or 25(OH)D2, or a combination of 25(OH)D3 and 25(OH)D2 is dependent on the level of endogeneous vitamin D2 and/or vitamin D3 per se of the person treated, preferably dependent on the amounts of vitamin D2 ingested and/or the amounts of vitamin D3 ingested and/or generated, respectively, by the treated persons.

Another object of the invention is a method of treating a vitamin D insufficiency or deficiency in obese persons, persons having the tendency to get obese and in persons having a history of malabsorption of vitamin D or persons with a genetic disposition to develop obesity, preferably in obese persons, and in persons having the tendency to get obese, more preferably in obese persons, with a vitamin D supplement, viz. a medicament, a nutraceutical or a food additive comprising (i) a combination of 25(OH)D3 and/or 25(OH)D2 and vitamin D2; or (ii) a combination of 25(OH)D3 and/or 25(OH)D2 and vitamin D3; or (iii) a combination of 25(OH)D3 and/or 25(OH)D2 and a combination of vitamin D2 and vitamin D3.

In another aspect the said use is characterized in that the ratio of the administered amount of vitamin D2, vitamin D3, or their combined amounts to the administered 25 hydroxy form(s) of the vitamin D is in the range between 1 to 5 and 5 to 1, between 1 to 4 and 4 to 1 or between 1 to 3 or 3 to 1, preferentially between 1 to 2 and 2 to 1, or in a ratio of 1 to 1, or in a ratio selected from the group of 1 to 1, 1 to 1.5, 1 to 2, 1.5 to 1.

In another aspect the said use is characterized in that the ratio of the administered amount of vitamin D2, vitamin D3, or their combined amounts to the administered 25 hydroxy form(s) of the vitamin D is in the range between 1 to 3 or 3 to 1, between 1 to 2 and 2 to 1, or preferentially in a ratio of 1 to 1, or in a ratio selected from the group of 1 to 1, 1 to 1.5, 1 to 2, 1.5 to 1.

In one aspect the said method is characterized in that the ratio of the administered amount of vitamin D2, vitamin D3, or their combined amounts to the administered 25 hydroxy form(s) of the vitamin D is in the range between 1 to 5 and 5 to 1, preferentially between 1 to 2 and 2 to 1, or in a ratio of 1 to 1, or in a ratio selected from the group of 1 to 1, 1 to 1.5, 1 to 2, 1.5 to 1.

In another aspect the said method is characterized in that the body mass index (BMI) of the person is determined and the ratio of the 25-hydroxy form(s) of the vitamin D2 or vitamin D3 to vitamin D2 and/or vitamin D3 is optimized based on the body mass index (BMI) of the person and/or the lifestyle of the person.

Said methods and uses are preferably for human persons, preferably in order to establish or maintain an adequate vitamin D status of said persons.

Another object of the invention is a composition comprising a unit dosage form comprising a vitamin suppmement as described above, preferably a combination of 25(OH)D2 and 25(OH)D3; or (i) a combination of 25(OH)D3 and/or 25(OH)D2 and vitamin D2; or (ii) a combination of 25(OH)D3 and/or 25(OH)D2 and vitamin D3; or (iii) a combination of 25(OH)D3 and/or 25(OH)D2 and a combination of vitamin D2 and vitamin D3 and a pharmaceutically acceptable carrier.

In one aspect the said composition is characterized in that the ratio of the administered amount of vitamin D2, vitamin D3, or their combined amounts to the administered 25 hydroxy form(s) of the vitamin D is in the range between 1 to 5 and 5 to 1, preferentially between 1 to 2 and 2 to 1, or in a ratio of 1 to 1, or in a ratio of 1 to 1, or in a ratio selected from the group of 1 to 1, 1 to 1.5, 1 to 2, 1.5 to 1.

In another aspect the said composition is characterized in that the ratio of the administered amount of vitamin D2, vitamin D3, or their combined amounts to the administered 25 hydroxy form(s) of the vitamin D is in the range between 1 to 5 and 5 to 1, between 1 to 4 and 4 to 1 or between 1 to 3 or 3 to 1, preferentially between 1 to 2 and 2 to 1, or in a ratio of 1 to 1, or in a ratio of 1 to 1, or in a ratio selected from the group of 1 to 1, 1 to 1.5, 1 to 2, 1.5 to 1.

In another aspect the said composition is characterized in that the ratio of the administered amount of vitamin D2, vitamin D3, or their combined amounts to the administered 25 hydroxy form(s) of the vitamin D is in the range between 1 to 3 or 3 to 1, between 1 to 2 and 2 to 1, or preferentially in a ratio of 1 to 1, or in a ratio of 1 to 1, or in a ratio selected from the group of 1 to 1, 1 to 1.5, 1 to 2, 1.5 to 1.

In another aspect the composition comprising a unit dosage form comprising a vitamin supplement as described above is provided in form of a capsule, preferably a soft gel capsule.

In another aspect the said composition is a pharmaceutical product, such as an OTC product or a nutraceutical product, such as a food product specifically designed to cope with the risk of counterproductive or negative effects due to accumulation of comparatively higher amounts of vitamin D alone.

Another object of the invention is a kit comprising a pharmaceutical formulation each of 25(OH)D3 and vitamin D3 or vitamin D2, or a pharmaceutical formulation of a composition of 25(OH)D2 and vitamin D3 or vitamin D2, or a pharmaceutical formulation of a composition of 25(OH)D3 and 25(OH)D2 and vitamin D3 or vitamin D2 and instructions to co-administer the pharmaceutical formulation to a subject suffering from a vitamin D deficiency/insufficiency, characterized in that the ratio of vitamin D3 and/or vitamin D2 to 25(OH)D2 or of vitamin D3 and/or vitamin D2 to 25(OH)D3 is in the range between 1 to 5 and 5 to 1, between 1 to 4 and to 1, between 1 to 3 and 3 to 1, preferentially between 1 to 2 and 2 to 1, or in a ratio of 1 to 1, or in a ratio selected from the group of 1 to 1, 1 to 1.5, 1 to 2, 1.5 to 1.

In a preferred embodiment the said range is between 1 to 3 and 3 to 1, between 1 to 2 and 2 to 1, or in a ratio of 1 to 1, or preferably in a ratio selected from the group of 1 to 1, 1 to 1.5, 1 to 2, 1.5 to 1.

In one aspect the said kit comprising a pharmaceutical formulation each of 25(OH)D3 and vitamin D3 or vitamin D2, or a pharmaceutical formulation of a composition of 25(OH)D2 and vitamin D3 or vitamin D2, or a pharmaceutical formulation of a composition of 25(OH)D3 and 25(OH)D2 and vitamin D3 or vitamin D2 and instructions to co-administer the pharmaceutical formulation to a subject suffering from a vitamin D deficiency/insufficiency, is characterized in that the ratio of vitamin D2, vitamin D3, or their combined amounts to the 25.hydroxy form(s) of the vitamin D is in the range between 1 to 5 and 5 to 1, between 1 to 4 and 4 to 1 or between 1 to 3 or 3 to 1, preferentially between 1 to 2 and 2 to 1, or in a ratio of 1 to 1, or in a ratio selected from the group of 1 to 1, 1 to 1.5, 1 to 2, 1.5 to 1.

In a preferred embodiment the said range is between 1 to 3 and 3 to 1, between 1 to 2 and 2 to 1, or in a ratio of 1 to 1, or preferably in a ratio selected from the group of 1 to 1, 1 to 1.5, 1 to 2, 1.5 to 1.

In another aspect of the invention the said kit comprising a pharmaceutical composition in accordance with this invention includes instructions on how the vitamin D supplement is to be administered.

Another aspect of the invention is a composition comprising a vitamin D supplement for use in the treatment of and/or prophylaxis of vitamin D insufficiency or deficiency in obese persons, overweight persons having the tendency to get obese or persons having a history of malabsorption of vitamin D or persons with a genetic disposition to develop obesity, characterized in that the said composition comprises a unit dose of vitamin D and 25(OH)D in a dosage for daily administration selected from the group as follows:
(i) For slightly obese individuals of unknown or unreliably known level of 25(OH)D in blood, respectively, who have a normal lifestyle with respect to UV-light exposure and daily uptake of vitamin D, the dose comprises vitamin D and 25(OH)D in a ratio of 1 to 1 and in amounts at the lower end of the range of 5 to 7.5 μg of each of the two products;
(ii) For slightly obese individuals of unknown or unreliably known level of 25(OH)D in blood, respectively, who have an unfavorable lifestyle with respect to UV-light exposure and daily uptake of vitamin D, the dose comprises vitamin D and 25(OH)D in a ratio of 1 to 1 and in amounts at the higher end of the range of 5 to 7.5 μg of each of the two products;
(iii) For slightly obese individuals of reliably known and not much too low level of 25(OH)D in blood (>20 to 30 ng/mL) the dose comprises vitamin D and 25(OH)D in a ratio of 1 to 1 and in amounts in the range of 5 to 7.5 μg of each of the two products;
(iv) For slightly obese individuals of reliably known and clearly low level of 25(OH)D in blood (>10 to 20 ng/mL) the dose comprises vitamin D and 25(OH)D in a ratio of 1 to 1.5, keeping the dose of vitamin D at 5 to 7.5 μg and raising the dose of 25(OH)D to 7.5 to 11 μg;
(v) For slightly obese individuals of reliably known and very low level of 25(OH)D in blood (≤10 ng/mL) the dose comprises vitamin D and 25(OH)D in a ratio of 1 to 2, keeping the dose of vitamin D at 5 to 7.5 μg and raising the dose of 25(OH)D to 10 to 15 μg;
(vi) For clearly obese individuals of unknown or unreliably known level of 25(OH)D in the blood, respectively, who have a normal lifestyle with respect to UV-light exposure and daily uptake of vitamin D, the dose comprises vitamin D and 25(OH)D in a ratio of 1 to 1 and in amounts at the lower end of the range of 7.5 to 11 μg of each of the two products;
(vii) For clearly obese individuals of unknown or unreliably known level of 25(OH)D in the blood, respectively, who have an unfavorable lifestyle with respect to UV-light exposure and daily uptake of vitamin D, the dose comprises vitamin D and 25(OH)D in a ratio of 1 to 1 and in amounts at the higher end of the range of 7.5 to 11 μg of each of the two products;
(viii) For clearly obese individuals of reliably known and not much too low level of 25(OH)D in blood (>20 to 30 ng/mL) the dose comprises vitamin D and 25(OH)D in a ratio of 1.5 to 1, keeping the dose of vitamin D at 7.5 to 11 μg, which results in a dose of 25(OH)D of 5 to 7.5 μg;
(ix) For clearly obese individuals of reliably known and clearly low level of 25(OH)D in blood (>10 to 20 ng/mL) the dose comprises vitamin D and 25(OH)D in a ratio of 1 to 1 and in amounts of 7.5 to 11 μg of each of the two products;
(x) For clearly obese individuals of reliably known and very low level of 25(OH)D in blood (≤10 ng/mL) the dose comprises vitamin D and 25(OH)D in a ratio of 1 to 1.5, keeping the dose of vitamin D at 7.5 to 11 μg, which results in a dose of 25(OH)D of 11 to 17 μg;
(xi) For extremely obese individuals of unknown or unreliably known level of 25(OH)D in blood, respectively, who have a normal lifestyle with respect to UV-light exposure and daily uptake of vitamin D, the dose comprises vitamin D and 25(OH)D in a ratio of 1 to 1 and in amounts at the lower end of the range of 11 to 15 μg of each of the two products;
(xii) For extremely obese individuals of unknown or unreliably known level of 25(OH)D in blood, respectively, who have an unfavorable lifestyle with respect to UV-light exposure and daily uptake of vitamin D, the dose comprises vitamin D and 25(OH)D in a ratio of 1 to 1 and in amounts at the higher end of the range of 11 to 15 μg of each of the two products;
(xiii) For extremely obese individuals of reliably known and not much too low level of 25(OH)D in blood (>20 to 30 ng/mL) the dose comprises vitamin D and 25(OH)D in a ratio of 1.5 to 1, keeping the dose of vitamin D at 11 to 15 μg, which results in a dose of 25(OH)D of 7.5 to 10 μg;
(xiv) For extremely obese individuals of reliably known and clearly low level of 25(OH)D in blood (>10 to 20 ng/mL) the dose comprises vitamin D and 25(OH)D in a ratio of 1 to 1 and in amounts of 11 to 15 μg of each of the two products;
(xv) For extremely obese individuals of reliably known and very low level of 25(OH)D in blood (≤10 ng/mL) the dose comprises vitamin D and 25(OH)D in a ratio of 1 to 1.5, keeping the dose of vitamin D at 11 to 15 μg, which results in a dose of 25(OH)D of 17 to 22.5 μg.

In connection with the above 15 particular dosage regimens the term “a dose of vitamin D and 25(OH)D” is preferably meant to be “a dose of vitamin D3 and 25(OH)D3”. In the case of using “a dose of vitamin D2 and 25(OH)D2”, or “a dose of vitamin D2 and 25(OH)D3”, or “a dose of vitamin D3 and 25(OH)D2”, or a dose of vitamin D2 or vitamin D3 and the combined 25-hydroxy forms thereof, (viz. 25(OH)D2 and 25(OH)D3) slight adaptions on the amount of each of individual components may be necessary. It is in the ambit of the skilled person to adapt the dosage of each of the two components under these circumstances.

Another aspect of the invention is a product package insert with instructions how to administer a composition comprising a vitamin D supplement for use in the treatment of and/or prophylaxis of vitamin D insufficiency or deficiency in obese persons, overweight persons having the tendency to get obese or persons having a history of malabsorption of vitamin D or persons with a genetic disposition to develop obesity in accordance with the above dosage regimen.

Another aspect of the invention is a kit comprising a composition as mentioned above and instructions on how the vitamin D supplement is to be administered.

As used herein, the singular forms “a,” “an,” and “the” include the plural.

The term “about” when used in reference to numerical ranges, cut-offs, or specific values is used to indicate that the recited values may vary by up to as much as 10% from the listed value. As many of the numerical values used herein are experimentally determined, it should be understood by those skilled in the art that such determinations can, and often will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

The term “unit dose” or “unit dosage” in connection with this patent application means an amount or unit of a vitamin D supplement that is separately identifiable as a pre-packed medication and in a form that is separately administered to or taken by an individual or patient, preferably an obese patient, such as e.g. a soft-gel capsule, or a liquid container configured for metered dosing (e.g. with a pipette or by spraying) whereby such unit dose is preferably to be taken daily until the vitamin D level is back to normal. It is noted that exemplary dosage forms as recited herein are not meant to be construed as limiting. Preferably the said unit dose or unit dosage is separately packed with a label specifying the amount of the vitamin D and/or 25(OH)D contained therein. This dosage form may contain the daily dosages as described or contain weekly dosages obtained by 7-fold increase of the number of dosages as described, respectively. Alternatively, monthly dosages are packed together in the form of four weekly dosages. If desired a number of daily dosages are packed together in a box or container, whereby this box is labeled with the name of the manufacturer or distributor, an identifying lot number and, if applicable, the expiration date of the vitamin D supplement.

The term “required amount of 25(OH)D2 or 25(OH)3, or a combination of 25(OH)D2 and 25(OH)D3” is defined as being the amount of 25(OH)D2 and/or 25(OH)D3 which should be supplemented to the individual or patient based on the difference between the measured blood level of the 25-hydroxy form of vitamin D and the corresponding broadly accepted target blood level which is 30 ng/mL, whereby the particular observations regarding lifestyle with respect to UV-light exposure and daily uptake of vitamin D in connection with the particular optimized situations and dosages as listed under (i) to (xv) above should be considered.

The phrase “level of endogeneous vitamin D2 and/or vitamin D3 per se” is to be understood as representing the amount of vitamin D3 which has been generated in a subject due to the exposure of the skin to sun-light (sun-induced vitamin D) or eventually UV-lamps (e.g. with a UV lamp that emits ultraviolet radiation similar to sunlight (290 to 315 nm); for details see Chandra P, Wolfenden L. L., Ziegler T. R., Tian J, Luo M, Stecenko A. A., Chen T. C. Holick M. F. Tangpricha V. “Treatment of vitamin D deficiency with UV light in patients with malabsorption syndromes: a case series” Photodermatol. Photoimmunol. Photomed. (2007): 23(5) 179-185)) plus the amout of vitamin D3 and vitamin D2 which has been taken up with the diet or via supplementation and been deposited and/or is present in blood, respectively. This combined level of endogenous vitamin D2 and/or vitamin D3 may be approximately deduced from determining the blood level of 25(OH)D and/or by taking into account information on the person's lifestyle (including sun-exposure), supplementation, and diet. Thus, the blood level of 25(OH)D is comparatively stable and approximately reflects a person's level of endogenous vitamin D2 and/or vitamin D3—besides some direct intake of 25(OH)D, which generally plays a minor role. The blood level of vitamin D2 and/or D3, however, is variable, and it should be noted that it is not finally determined whether the respective average ground-level per se has a relevant additional impact besides its two main roles (Gibson C. C, Davis C. T., Zhu W., Bowman-Kirgin J. A, Walker A. E., Tai Z., Thomas K. R., Donato A. J., Lesniewski L. A., Li D. Y. “Dietary Vitamin D and Its Metabolites Non-Genomically Stabilize the Endothelium” PLOS ONE Oct. 15, 2015 pp. 1-15). Those two roles regard the well-described main role as precursor of 25(OH)D and the role vitamin D plays in adipocytes. Thereby the latter has up until today not been adequately considered by the experts in the field. It led the inventors to conclude that preferably a vitamin D insufficiency or deficiency in obese persons, persons having the tendency to get obese and in persons having a history of malabsorption of vitamin D or persons with a genetic disposition to develop obesity should be treated with a vitamin D supplement, viz. a medicament, a nutraceutical or a food additive comprising (i) a combination of 25(OH)D3 and/or 25(OH)D2 and vitamin D2; or (ii) a combination of 25(OH)D3 and/or 25(OH)D2 and vitamin D3; or (iii) a combination of 25(OH)D3 and/or 25(OH)D2 and a combination of vitamin D2 and vitamin D3.

It should be understood that the term “level of endogenous vitamin D2 and/or vitamin D3” and even more so the respective blood level addresses averages of a rather varying values. In particular the blood level may be determined analytically, whereby a single measurement won't say too much. But it may approximately be characterized by combining information on the subjects' lifestyle, exposure to UV-radiation (e.g. sunlight or UV lamp), diet, 25(OH)D-level and the constitution of a person such as e.g. by the BMI of the person.

As understood herein, the term “obese person” is to be understood as encompassing obese persons, persons having the tendency to get obese, persons having a history of malabsorption of vitamin D, or persons with a genetic disposition to develop obesity. It is herein proposed to focus on the BMI (body mass index=body mass divided by the square of the body height; unless otherwise specified, BMI is understood as expressed in kg/m²) when discussing the ratio of supplementation of vitamin D and/or its major metabolite, 25(OH)D. One reason behind this proposal is that the main routes of up-take for vitamin D and/or its major metabolite 25(OH)D differ. As noted above vitamin D is mainly taken up by the body via the lymphatic pathway. On the other hand, 25(OH)D is mainly taken up through the entero-hepatic system. In this respect it is important to consider that the characteristics of the lymphatic system depend strongly on the BMI of the individual. Thereby we speak in this whole text of the BMI in the sense of the most practical and easy approach to approximately describing a person's “level of obesity”, aware of the fact that this level can be more accurately determined by more demanding procedures which, e.g., provide a true measurement of the person's ratio of fatty to muscular tissues. Thus, we do by no means want to exclude such more demanding procedures where we simply say BMI, and they may, if available, replace the determination of the BMI. However, we want to underline that a person's BMI is statistically the much more relevant parameter than the persons weight when attempting to make predictions regarding its up-take of vitamin D and/or 25(OH)D, respectively, from the gastro-intestinal tract. Still, the following holds true in this statistical sense: The higher the BMI, the slower the lymphatic flux, the higher the permeability of lymphatic vessels, and the higher the infiltration of macrophages into adipose tissues (Ellulu M S, Patimah I, Khaza'ai H, Rahmat A and Abed Y; Arch Med Sci (June 2017) 4: 851-863; document available under the link (last assessed Mar. 21, 2020) https://www.ncbi.nlm.nih.gov/pmc/articles/PMC5507106/).

It has been shown that obese subjects (BMI ≥30 kg/m²) require much higher vitamin D-dosages than non-obese to reach a healthy blood-level of 25(OH)D. Thus, the BMI-dependence of the lymphatic system's characteristics seemingly is a non-linear function. This raises doubts regarding the often-cited explanation that the higher requirements are simply due to the obese subjects' proportionally higher amount of adipose tissue. We hypothesized that there may be BMI-levels higher than about 30, for which the supplementation of 25(OH)D or combinations of 25(OH)D and vitamin D is advantageous and for which the sheer rising of the vitamin D dosage to get adequate blood levels of 25(OH)D may be counter-productive. Thus, achieving 25(OH)D blood levels >30 ng/mL by dosing vitamin D alone in cases of heavy obesity is expected to ask for dosages which are high enough in some types of adipocytes to generate levels of the vitamin and its metabolites, which promote reproduction and risk to aggravate obesity.

This point has already been addressed when discussing the effects of vitamin D and its metabolites upon the adipocytes themselves. Furthermore, as discussed herein we assume that vitamin D is mainly stored on the surface of the adipocytes and of their lipid droplets (see below for the detailed discussion of this assumption). Our proposed dosage scheme (as shown in Table 3 further below) derives from this assumption.

Besides being of value in view of the calculation of body fat percentages, weights, and approximate surfaces plus their distribution within the considered population, Tables 1 and 2 illustrate the high statistical importance of higher BMI individuals in some populations. Table 2 illustrates in addition that body weights and BMIs are correlated. This means that studies reporting the correlation of a given result with body weight, but not BMI, like the publication on loading doses of L. van Groningen et al. in Europ. J. of Endocrinology 2010, 162: 85-811, are likely to be applicable to a subgroup of the population only. It also means that it is reasonable to assume that the average BMI is increasing, if going from segments of lower to segments of higher average weight. This has been illustrated for US men in Tables 1 and 2 below, but it can be reasonably expected to hold for women or corresponding populations of other nations as well. Finally, it is clear to the skilled person that weights and BMIs should be recorded in order to draw obesity-related conclusions from supplementation studies.

The results summarized in Table 1 have been obtained based on intuitive estimates for US men, whereby available information on average weight, height, and BMI plus the published statement (https://dqydj.com/height-percentile-calculator-for-men-and-women/; assessed Feb. 12, 2020) that heights are symmetrically distributed around the mean, were considered. In addition, weights were assumed to be non-symmetrically distributed around the mean (steeper decrease towards the heavy end). The numerical values used and derived are compiled in which nicely reproduces the published averages:

TABLE 1
Assumed distribution [%]

| | | of height | | | | | |
|---|---|---|---|---|---|---|---|
| | | 5% | 15% | 30% | 30% | 15% | 5% |
| of weight | | 1.5 m | 1.6 m | 1.7 m | 1.8 m | 1.9 m | 2.0 m |
| 17.5% | Weights [kg] | 45 | 52 | 59 | 66 | 73 | 80 |
| | % of popul. | 0.875 | 2.625 | 5.25 | 5.25 | 2.625 | 0.875 |
| | BMI | 20 | 20.3 | 20.4 | 20.4 | 20.2 | 20 |
| 25% | Weights [kg] | 55 | 64 | 73 | 82 | 91 | 100 |
| | % of popul. | 1.25 | 3.75 | 7.5 | 7.5 | 3.75 | 1.25 |
| | BMI | 24.4 | 25 | 25.2 | 25.3 | 25.2 | 25 |
| 30% | Weights [kg] | 65 | 76 | 87 | 98 | 109 | 120 |
| | % of popul. | 1.5 | 4.5 | 9 | 9 | 4.5 | 1.5 |
| | BMI | 28.9 | 29.7 | 30.1 | 30.2 | 30.2 | 30 |
| 15% | Weights [kg] | 75 | 88 | 101 | 114 | 127 | 140 |
| | % of popul. | 0.75 | 2.25 | 4.5 | 4.5 | 2.25 | 0.75 |
| | BMI | 33.3 | 34.4 | 34.9 | 35.2 | 35.2 | 35 |
| 10% | Weights [kg] | 85 | 100 | 115 | 130 | 145 | 160 |
| | % of popul. | 0.5 | 1.5 | 3 | 3 | 1.5 | 0.5 |
| | BMI | 37.8 | 39.1 | 39.8 | 40.1 | 40.2 | 40 |
| 2.5% | Weights [kg] | 95 | 112 | 129 | 146 | 163 | 180 |
| | % of popul. | 0.125 | 0.375 | 0.75 | 0.75 | 0.375 | 0.125 |
| | BMI | 42.2 | 43.8 | 44.6 | 45.1 | 45.2 | 45 |

Explanation of Table 1 Together with Information to Generate it:
(i) height=1.75 m, published 1.757 m (see https://dqydj.com/height-percentile-calculator-for-men-and-women/; assessed Feb. 12, 2020).
(ii) weight=89.875, published 89.8 kg (see: https://www.healthline.com/health/mens-health/average-weight-for-men; assessed Feb. 12, 2020).
(iii) BMI=29.17, published ≈29 (see: https://en.wikipedia.org/wiki/List_of_countries_by_body_mass_index; assessed Feb. 12, 2020). Here, it shall be added that a BMI of 32.1 and of 21.8 has been reported for the average US male and the average male from India, respectively, by the WHO in 2014. Other sources (see: https://dqydj.com/bmi-percentile-calculator-men-women-united-states/assessed Feb. 12, 2020) report lower values for US men while an “average” of 29.1 is reported under (https://www.healthline.com/health/mens-health/average-weight-for-men#takeaway; assessed Mar. 21, 2020).
(iv) published statement on symmetrically distributed heights (https://dgydj.com/height-percentile-calculator-for-men-and-women/; assessed Feb. 12, 2020).

Remarks regarding: The table should not be interpreted in the sense that, e.g., 0.875% of the population are exactly 1.50 m tall and weigh exactly 45 kg. The figures are means meant to (in this example) cover heights between 1.45 and 1.55 m and weights between 40 and 50 kg. The bold %-age figures in the table plus reasonable assumptions regarding extreme BMIs were used to generate it.

In Table 2 the the figures from Table 1 are arranged in such a way as to show the BMI's weight-dependence. The percentages from Table 1 were thereby assigned to the weight- and BMI-ranges shown in Table 2 and were simply added up, if applicable.

TABLE 2

| BMI ranges | Weight ranges [kg] | | | | | | | | | |
|---|---|---|---|---|---|---|---|---|---|---|
| [kg/m²] | >40-50 | >50-60 | >60-70 | >70-80 | >80-90 | >90-110 | >110-130 | >130-150 | >150 | Sum (%) |
| 42.5-47.5 | | | | | | 0.125 | 1.125 | 0.75 | 0.5 | 2.5 |
| 37.5-42.5 | | | | | 0.5 | 1.5 | 6.0 | 1.5 | 0.5 | 10 |
| 32.5-37.5 | | | | 0.75 | 2.25 | 4.5 | 6.75 | 0.75 | | 15 |
| 27.5-32.5 | | | 1.5 | 4.5 | 9.0 | 13.5 | 1.5 | | | 30 |
| 22.5-27.5 | | 1.25 | 3.75 | 7.5 | 7.5 | 5.0 | | | | 25 |
| 17.5-22.5 | 0.875 | 7.875 | 5.25 | 3.5 | | | | | | 17.5 |
| Sum (%) | 0.875 | 9.125 | 10.5 | 16.25 | 19.25 | 24.625 | 15.375 | 3.0 | 1.0 | 100 |
| Φ BMI | 20 | 20.68 | 23.21 | 25.77 | 28.90 | 30.58 | 37.20 | 40 | 42.5 | |

Remarks rewarding Table 2: The approximate average BMIs shown at the bottom were calculated in consideration of the percentages from Table 1 and based on the arithmetic mean of the BMI-ranges indicated (e.g. 25.0 as mean of 22.5-27.5). The overall approximate BMI-average obtained accordingly is 29.12 and differs no more than marginally from the 29.17 reported above. The shading highlights the fields containing the highest population percentages.

In the following the preferred methods and uses in accordance with the present invention are described. For an easier understanding it is referred to the figures which should not be construed as limiting the invention in any way.

FIG. 1 shows the weight-dependence of the BMI as expected for the complete population of adult US men (compare Table 2 above). The vertical bars show the BMI-ranges expected to be applicable to subjects in the respective weight groups. The position of the circles on their respective bar reflects the weighed mean BMI. The size of the circles visualizes the percentage of all subjects belonging to the respective weight group. That the function shown is flat in the beginning and towards the end, but steeper in between, is due to the fact that there is a practical lower and upper end of the distribution of heights and weights.

FIG. 2 shows a schematic representation of the clinical study showing the number of healthy participants and patients with fat malabsorption screened and randomized in the two arms of the study.

FIG. 3a shows the mean (±SEM) change in serum vitamin D3 concentration (ng/mL) versus time curve (in hours) after administration of a single dose of oral 900 μg vitamin D3 in healthy participants (N=10; solid line) and patients with fat malabsorption (N=6; dotted line).

FIG. 3b shows the mean (±SEM) change in serum 25-hydroxyvitamin D3 concentration (ng/mL) versus time curve (in hours) after administration of a single dose of oral 900 μg 25-hydroxyvitamin D3 in healthy participants (N=10; solid line) and patients with fat malabsorption (N=6; dotted line).

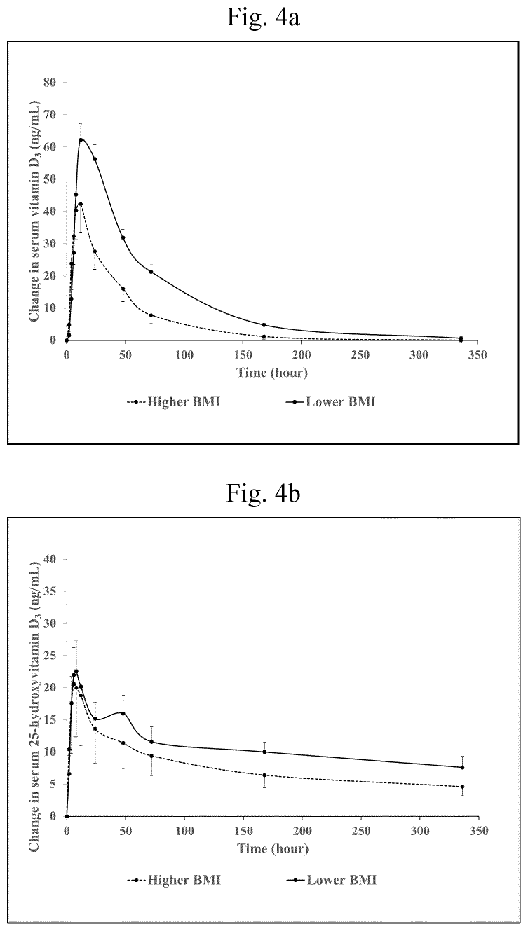

FIG. 4a shows the mean (±SEM) change in serum vitamin D3 concentration versus time curve after single dose of oral 900 μg vitamin D3 in healthy participants with higher body mass index (N=5; dotted line) and lower body mass index (N=5; solid line).

FIG. 4b shows the mean (±SEM) change in serum 25-hydroxyvitamin D3 concentration versus time curve after single dose of oral 900 μg 25-hydroxyvitamin D3 in healthy participants with higher body mass index (N=5; dotted line) and lower body mass index (N=5; solid line).

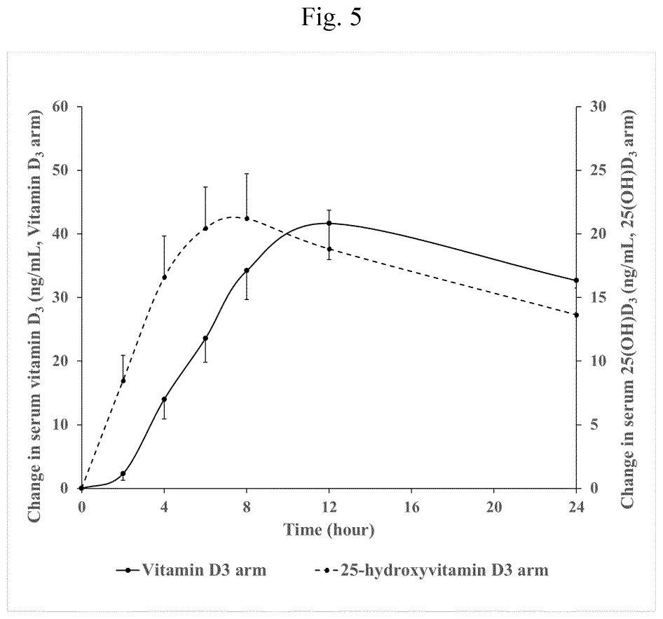

FIG. 5 shows the change in serum concentration of vitamin D3 (left scale) and 25(OH)D3 (right scale) in dependence of the time after the oral administration of 900 μg vitamin D3 (solid line) and 900 μg 25(OH)D3 (dotted line), respectively. The peak of the serum concentration is reached after about 12 hours for vitamin D3 and 8 hours for 25(OH)D3.

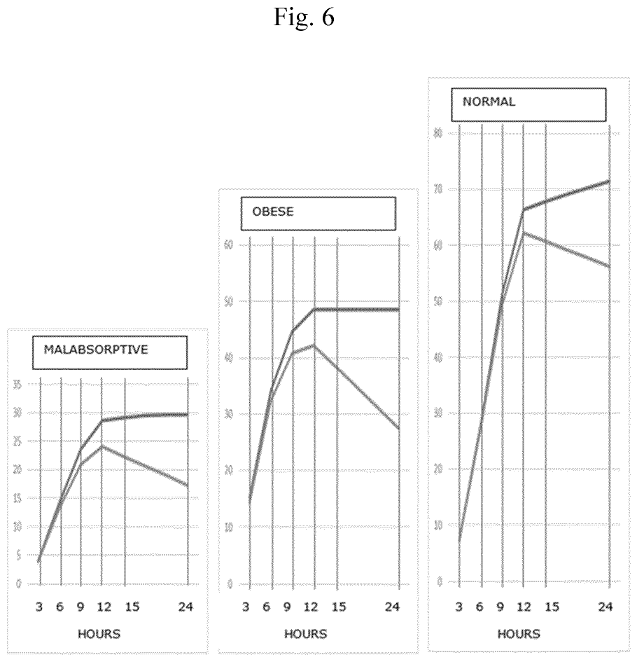

FIG. 6: The upper curves show the calculated total amounts of vitamin D3 arriving in blood of malabsorptive, obese and normal individuals. The lower curves show the experimental blood levels of vitamin D3 expressed in ng/mL of blood. The upper curves are as well expressed in ng/mL of blood, disregarding differing total blood volumes for malabsorptive, obese and normal individuals. The Figure shows that the vitamin D level in blood as derived from the respective fitted experimental blood level curves (concentration versus time) by assuming that deposition (mainly in fatty tissues) starts to play a role as soon as vitamin D3 appears and accordingly reduces the observed levels. The further assumptions used to get the calculated curves were the following:
The rate of deposition is proportional to the concentration present in blood at any time.
This concentration-dependence doesn't change up to the point when the uptake is completed.
At that point the concentration-dependent slope of the curve reaches a (negative) maximum.
This slope can be used to calculate the increments of substance, which were per small time-period deposited, before they resulted in an observable blood level, and the increments which were measured at the respective time-period's start, but deposited in its course.
Adding those increments to the observed blood levels delivers the respective curves describing the calculated total of substance which actually arrived (i.e., was deposited).

Of course, the difference between calculated arrival and observed blood level describes just disappearance in the course of the uptake and the short subsequent phase. It does not per se distinguish disappearance due to deposition and other body functions such as, e.g., metabolism.

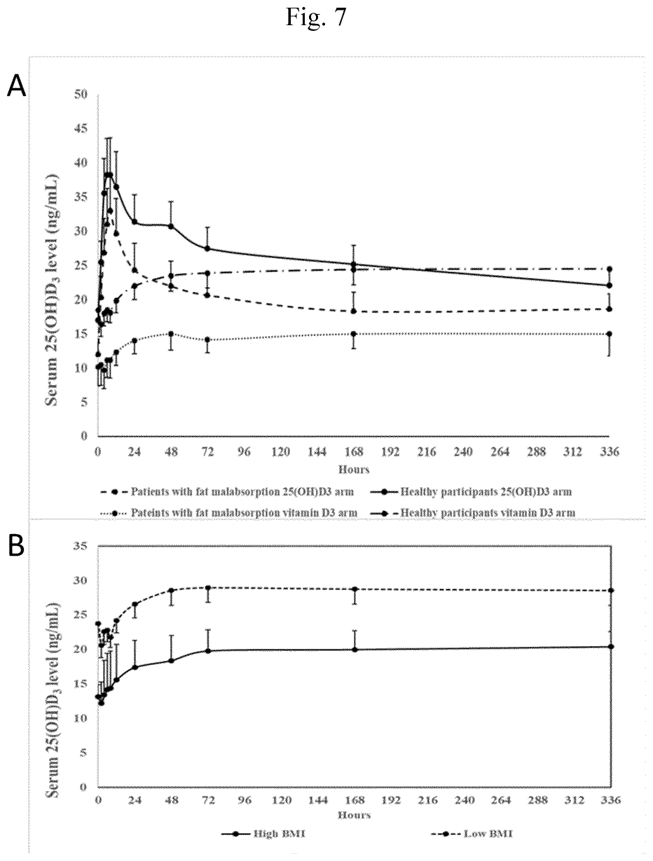

FIG. 7A shows the mean (±SEM) serum 25-hydroxyvitamin D3 concentration (ng/mL) versus time curve (in hours) observed after administration of a single dose of oral 900 μg vitamin D3 in healthy participants (N=10; line interrupted by dots) and patients with fat malabsorption (N=6; lowest dotted line). Also shown for comparison are the mean (±SEM) serum 25-hydroxyvitamin D3 concentrations (ng/mL) as obtained after administration of a single dose of oral 900 μg 25-hydroxyvitamin D3 to the same subjects.

FIG. 7B shows the mean (±SEM) serum 25-hydroxyvitamin D3 concentration (ng/mL) versus time curve observed after administration of a single dose of oral 900 μg vitamin D3 in healthy participants with higher body mass index (N=5; solid line) and lower body mass index (N=5; dotted line).

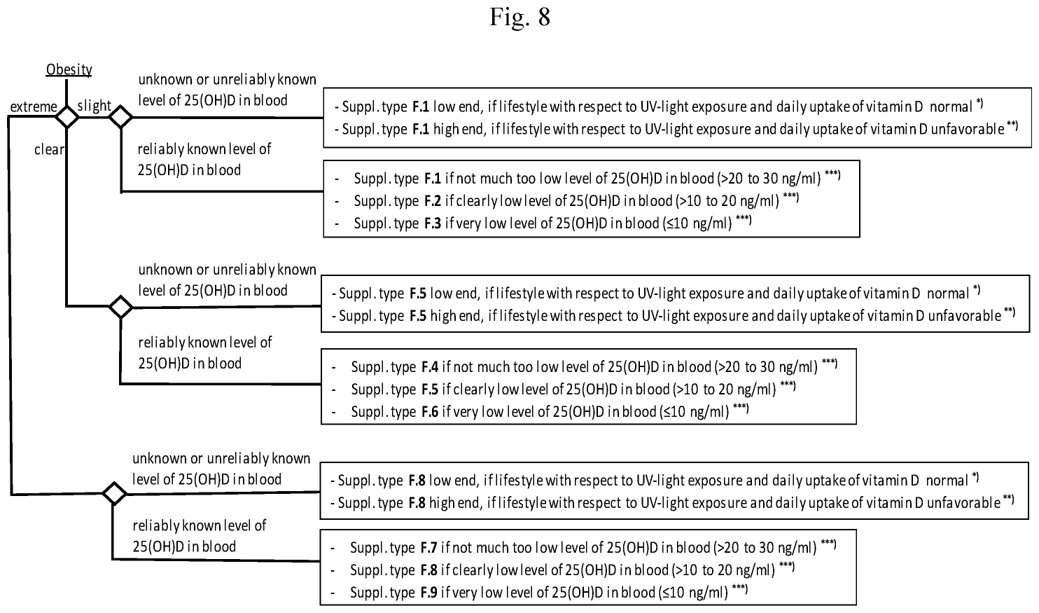

FIG. 8 shows a decision tree (flow chart) related to Table 3 (cp. below). Together with Table 3 it provides guidance regarding the dosing of combinations of vitamin D and 25(OH)D in function of the level of obesity of the person in question and the person's vitamin D status, viz. blood level of 25(OH)D, provided this level is known reliably enough. In addition, it provides guidance on how the person's constitution and generalized lifestyle can be used to approximately determine an optimized dosage of the combination, if reliable analytical information on the person's vitamin D status is unavailable. In this figure the following footnotes are to be considered:

*) The dose may be lowered further and/or vitamin D may be left out completely in case of an extreme favorable life style etc.

**) Move from F.1 to F.2, from F.5 to F.6, and from F.8 to F.9, repectively, if the person has a very unfavorable lifestyle etc. and is at the upper end of the respective BMI-range and is very tall.

***) Dosages may be adapted beyond the respective range, in case of analytical results far outside of the ranges generally observed for the respective BMI-group. E.g., vitamin D may be raised beyond 15 μg in case of heavily inflammatory obesity.

If considering the experience, e.g., that subjects with a BMI of about 35 (viz. a BMI between 32.5 and 37.5) need in the average roughly 2 to 3 times higher supplementation of vitamin D than subjects with a BMI of about 25 (i.e a BMI between 22.5 and 27.5) and if using Table 2 to define corresponding “normal groups” (of US men) one finds the following weighed average weights: For the BMI 25 group 80.5 kg and for the BMI 35 group 107.5 kg. In other words: The weight-increase is 33.5%, and the BMI-increase is 40%. None of the two values “explains” the more than doubled vitamin D-demand, and therefore additional factors must be considered.

The van Groningen et al. publication cited above recommends administering the total “loading dose” over many weeks and underlines that the required dosage depends on the body weight (BW) which appears in the formula, but not the BMI—so that no correlation with the BMI was found. However, van Groningen et al. do not address the question whether and how the initial level of insufficiency or deficiency was correlated with the BMI. In addition, the publication explicitly excludes the application of the formula in case of obesity which is defined by van Groningen et al. as being a BW >120 kg (!).

It would be surprising, if obese individuals who easily absorb lipids from their guts had a diminished absorption of vitamin D, while it is conceivable that they have in the average a reduced 25-hydroxylation activity of their liver. Our observation that much less of the dosed vitamin D3 per se appears in the blood of obese people than in non-obese people may, therefore, have to be interpreted as follows: vitamin D3 enters the body mainly via the lymphatic pathway, and the part not appearing in the blood of obese is “lost” elsewhere, particularly in adipose tissue.

It is assumed in the following that about ⅔ of the vitamin D administered to normal individuals is rather rapidly forming 25(OH)D, while FIG. 4a actually shows that their vitamin D3 was rapidly falling to 41% of Cₘₐₓ and FIG. 7B shows that there was no equivalent increase of 25(OH)D3. However, this is not implying 59% of essentially irreversible deposition. At the contrary, there is probably a certain percentage of non-adipose deposition, which is higher than in obese subjects, and there is comparatively faster release from adipocytes. Accordingly, the assumption that normal individuals are much less slowly than obese individuals converting about ⅔ of the vitamin D to 25(OH)D remains credible However, it should be noted that blood levels of 25-hydroxy vitamin D only gradually increase after an oral dose of vitamin D. This is because it is believed that the vitamin D is incorporated into adipocytes/fat and slowly released back into the circulation and being converted to 25(OH)D.

The followed time/concentration-curves after administration to obese, normal, and malabsorptive individuals, respectively, of a single high oral dose of vitamin D3 (p.o.) as presented in the Examples part further below show distinctly differing overall shapes (see FIGS. 3A and 4A). Analytical determination of vitamin D3 per se in the blood samples collected leads to the following observations:
The curve obtained for the healthy normal BMI subjects rises quickly after dosing and reaches the highest initial level (FIG. 4a). This level decreases rather steeply, but only with a certain delay after Cₘₐₓ (the maximum concentration achieved).
The curve for the healthy obese subjects rises even slightly faster than the curve for normal ones, but reaches a lower Cₘₐₓ, and the subsequent decrease follows without delay, quickly displaying an even steeper slope (FIG. 4a).
The curve for the malabsorptive subjects reaches a much lower Cₘₐₓ (FIG. 3a).

In order to further analyze the results, the shape of the concentration/time-curves describing the observed average blood levels of vitamin D3 was used to approximately calculate its total uptake into blood after dosage. The calculation was based on the assumptions compiled in the comments regarding FIG. 6. Metabolism in terms of the appearance of 25(OH)D3 which was experimentally determined in parallel to vitamin D3 can thereby be regarded as part of the immediately disappearing percentage of the total uptake into blood, and it is reasonable to assume that there is no other relevant metabolic step in this early phase, which has to be taken into account.

FIG. 6 presents the accordingly calculated total uptake and the experimentally determined levels of vitamin D3 for the first 24 hours after dosing. The difference between the upper (calculated) and the lower (experimental) curve represents the sum of metabolism and deposition. Herein the assumption is made that the elimination can be disregarded, because as known to the skilled person there is essentially no elimination of non-metabolized vitamin D3 from blood into urine. Metabolism in the first 24 hours results in all three cases (viz., malabsorptive, obese, and normal subjects) in the consumption of about 2 to 5 ng/mL, viz. a small part of the mentioned calculated difference (compare FIG. 7). No correction relating to said consumption seems necessary for determining the metabolite's observed concentration because the metabolite is rapidly forming a DBP-complex, characterized by a long half-life. However, the fact that the 25(OH)D3 formed must be determined as difference to baseline adds an element of uncertainty and the assumption that no more than a minor part of the 25(OH)D3 formed in this phase may not be absolutely to the point.

When comparing deposition in healthy obese and normal low BMI subjects, one finds
at the time point of 15 hours 1.5 times more and
at the time point of 24 hours 1.48 times more deposition in the healthy obese,

wherein the same correction regarding metabolism, as described above, was used for the obese and for the normal individuals, viz. 1.5 ng/mL at 15 hours and 3.5 ng/mL at 24 hours, respectively. These values come from FIG. 7B and are charged with a significant uncertainty—due to the significant experimental error in the early part of the respective curves. But they make sense, if inspecting the subsequent increase which can be more reliably determined.

Thus, if assuming a total blood volume of 5 liters in both cases, the healthy obese subjects deposited in the first 24 hours 92.5 μg and the normal subjects 62.5 μg of vitamin D3. The following was considered in view of calculating the mean amounts of body fat of the two groups: The average BMI of the subjects, their average age (32+/−2.7 years) and their gender (8 females, 2 males). It was assumed that they have average height (1.7 m). The published formulae:

(https://www.calculator.net/body-fat-calculator.html—cp. P. Deurenbeg et al., Br. J. Nutr. 65(2), 105-14 (1991)) cited here was used to derive body fat percentages (BFP) from the BMIs:

Body Fat Percentage (BFP) Formula for Adult Males:

BFP=1.20×BMI+0.23×Age−16.2

Body Fat Percentage (BFP) Formula for Adult Females:

BFP=1.20×BMI+0.23×Age−5.4

These calculations yield the following results:
for the normal BMI group BFP=26.92%, which results in 17.58 kg body fat, and
for the healthy obese group BFP=37.48%, which results in 34.01 kg body fat.

The ratio “mass/mass” of body fat of the two groups is R_{M}=1.935 and the ratio “surface/surface” of the body fat is R_{S}=R_{M}^{2/3}=1.553 (assuming congruence, in particular similar distribution of sizes and dimensions of adipocytes), viz. close to the about 1.50 reported here for deposition. This may be interpreted that the given dose of vitamin D3 resulted after 24 hours in a comparable load of the relevant surfaces in and on adipocytes of our low and high BMI subjects. The present inventors have discovered this surprising relationship, which is important when defining upper limits for vitamin D per se and optimized combinations 25(OH)D plus vitamin D to be supplemented to individuals who are overweight or obese or have a tendency to get obese.

The two discussed curves in FIG. 4a come to a turning point after about 2% days. This is very likely the point where the dominating role of deposition comes to an end. Here, the blood level of the low BMI subjects has fallen to ≈41% and the one of the high BMI subjects to ≈23% of Cₘₐₓ, translating in absolute terms to similar deposited amounts in both cases.

Thereby this present discussion, the slow-down of metabolism in obesity (including 24-hydroxylation of 25(OH)D), the difference in blood volume, the in the average larger size of adipocytes and of lipid droplets in obesity, and the low BMI subjects' comparatively increased deposition in non-fatty tissues strongly suggest that the high and the low BMI subjects achieved similar density of vitamin D3 on the critical surfaces in fatty tissue.

The following relates to the coverage of the surface of adipocytes with vitamin D3. Referring to the publication by Karin G. Stenkula et al. (Am J Physiol Regul Integr Comp Physiol 315: R284-R295, 2018) on adipose cell size and reasonably considering the size-distribution reported there, the percentage of the relevant surfaces on and in adipocytes, which has been covered by no more than a mono-molecular layer of vitamin D as deposited in our low BMI group after 2% days, has been calculated. A homogenous cell diameter of 50 μm was determined to deliver about the same overall cell-surface one calculates by working with the rather wide experimental distribution of cell sizes as reported by Karin G. Stenkula et al. Thereby it was in the sense of a model consideration assumed that cubes containing large lipid droplet cubes can be used to describe the cells' shape (in other words: cubes of an edge-length of 50 μm enclosing cubes of about the same size were determined to represent a reasonable model for the description of the combined surface of the variable size cells' with their lipid droplets). In addition, it was assumed—in line with the observations reported in the already cited work of Susanne Prattes et al. (Journal of Cell Science 113, 2977-2989 (2000) https://jcs.biologists.org/content/joces/113/17/2977.full.pdf) that the critical surfaces consist in the external and the internal side of the cell membrane and the surface of the lipid droplets (all three assumed to have about the same size). Working with 17.6 kg of fatty tissue, with a deposition of approximatively 7400 I.U. of vitamin D3 after 2½ days, and with a surface of approximatively 2 nm² covered by a single molecule of vitamin D3 resulted in about 0.01% surface coverage. The following can be concluded: On the one hand, it is not necessary to assume migration of vitamin D into lipid droplets to explain the observations made. On the other hand, the adipocytes' capacity to pick-up vitamin D without impacting its cellular physiology is not endless.

The lasting increase with regard to baseline by about 5 ng/mL of the blood level of 25(OH)D3 in low and high BMI subjects (FIG. 7), wherein observed effects for both groups are comparable, as observed after administering the same high dose of vitamin D3, supports the assumption of very similar critical surface densities of vitamin D3 in the fatty tissues of both groups. The general slow-down of metabolism in obesity explains the slower initial increase of the level of 25(OH)D3 in the high BMI individuals. The fact that the high dose of vitamin D3 does not fully compensate the obese subjects' 25(OH)D-deficit, supports our proposition to combine vitamin D and 25(OH)D. It may be hypothesized that there is at least some control of the deposition-dissolution-equilibrium in adipocytes, which prevents high levels of dissolved vitamin D in order to avoid excessive formation of metabolites within the adipocytes.

Administering a high dose of vitamin D3 led after 24 hours to a ratio R of about 1.5 times higher deposition in case of the higher BMI subjects. Thereby R is herein understood as the ratio of deposition between the higher BMI subjects and normal BMI subjects. Thus, the ratio R is approximately the amount of vitamin D (in the experimental case described herein D3) leaving the blood and being deposited in high BMI subjects divided by the amount of vitamin D leaving the blood and being deposited in low BMI subjects. In this connection the term “approximately” is used because the actual observable is the respective concentration of vitamin D present in blood at the beginning and the end of the period of observation. This means that metabolism is neglected (introducing a minor error, because deposition is in the discussed early phase much more important than metabolism and because the metabolic activity in high and low BMI subjects is rather similar). In addition, one has to consider the total blood volume (which is somewhat higher in the high BMI subjects) when calculating amounts on the basis of concentrations in the blood.

After 2½ days the relevant impact of deposition comes to an end. Deposition-dissolution-equilibria and metabolism take over. The ratio regarding total deposition in obese versus non-obese cannot anymore be reasonably approximated based on the approach used to determine the total uptake into blood for the initial phase of the concentration/time-curves. Working just with the difference to Cₘₐₓ and considering the differing blood volumes results at this point in R=1.05 times higher deposition in fatty tissues in the high BMI case (again disregarding differences of metabolism, which play a minor role).

Just for the sake of clarity it is emphasized that non-identical ways were used to calculate the above two R values for 24 hours and for 2½ days, respectively.

Fatty tissues play the dominant role regarding vitamin D deposition, in particular in obese persons. To neglect deposition in other tissues would, however, be inaccurate, particularly when discussing the fate of 25(OH)D and 1,25(OH)D2. Deposition of 25(OH)D and 1,25(OH)D2 must be considered when interpreting the steady (viz. to be understood as constant) level of 25(OH)D3 as observed in our experiments for two weeks after administering vitamin D3. It must be considered as well when deciding to supplement vitamin D and 25(OH)D, viz. two related pro-hormones together, in carefully adjusted amounts. This combined dosage of vitamin D and 25(OH)D in specific amounts has now been found to be better than dosing larger amounts of vitamin D, in particular vitamin D3, to obese individuals, wherein vitamin D3 is loaded into adipocytes. According to the present inventors, the goal is to achieve a vitamin D surface density on the adipocytes of obese individual that matches the ideal surface density in normal, non-obese individuals.

According to the finding that the identical dose of vitamin D3 likely results in comparable vitamin D surface density in adipocytes of two (vitamin D3 insufficient or vitamin D3 deficient) groups of individuals differing by roughly 10 BMI-units, it can be considered unlikely that the R ratio as defined herein strongly changes with the dose of vitamin D, in particular when reducing the dose of vitamin D.

It is further stipulated that achieving the targeted optimum surface density of vitamin D in and on adipocytes of vitamin D insufficient or deficient obese individuals will generally not normalize their vitamin D status in terms of blood level of 25(OH)D. According to the present invention, this deficit is preferably compensated by supplementing 25(OH)D in addition.

The dose of 25(OH)D to be supplemented in addition to the adipose tissues' limited needs for vitamin D depends on the individual's level of insufficiency or deficiency and to an extent on the formulation of 25(OH)D employed. Further parameters which play a certain role regard, e.g., gender, age, and lifestyle (including diet). Thereby in particular the lifestyle impacts the level of insufficiency or deficiency, besides impacting obesity. There aren't many reliable studies considering these parameters and linking dosages of 25(OH)D with the vitamin D status achieved.

The following Table 3 summarizes optimized conceivable supplementation schemes according to the present invention for healthy obese individuals displaying frequently encountered levels of vitamin D insufficiency or deficiency. It differs from the prior art in particular with regard to the low dosages of vitamin D per se from what has up to today been broadly advocated or tolerated, respectively. It further departs from cautious vitamin D3 supplementation recommendations for vitamin D insufficient normal adults (BMI ≈25 kg/m²—normal supplementation p.o.=200-600 I.U./day). According to the present invention, depending, i.a., on the person's lifestyle, the dosages in the table may be supplemented throughout the year or just in the months of reduced sun-exposure. The daily doses shown may be translated to equivalent weekly dosage schemes. Table 3 disregards specific malabsorptive patients (e.g. after bariatric surgery). It is applicable for healthy obese as well as unhealthily obese individuals, in particular in connection with the footnotes accompanying the decision tree shown in FIG. 8 (see corresponding legend) and explained below which adresses, e.g., cases of extremely unhealthy (viz. inflammatory) obesity. However, dosage schemes should in those latter cases not be decided upon without consulting a health professional.

TABLE 3
Recommended daily administration of vitamin D supplements.

| | | Initial deficiency/insufficiency (25(OH)D in ng/mL of blood) | | | | | |
|---|---|---|---|---|---|---|---|
| Level of | | >20 to 30 | | >10 to 20 | | ≤10 | |
| obesity | | Contents | Ratio | Contents | Ratio | Contents | Ratio |
| (BMI) | Components | [μg] | D/25(OH)D | [μg] | D/25(OH)D | [μg] | D/25(OH)D |
| Slight | Vit. D | 5-7.5 | 1/1 | 5-7.5 | 1/1.5 | 5-7.5 | 1/2 |
| (30-36) | 25(OH)D | 5-7.5 | (F.1) | 7.5-11 | (F.2) | 10-15 | (F.3) |
| Clear | Vit. D | 7.5-11 | 1.5/1 | 7.5-11 | 1/1 | 7.5-11 | 1/1.5 |
| (37-43) | 25(OH)D | 5-7.5 | (F.4) | 7.5-11 | (F.5) | 11-17 | (F.6) |
| Extreme | Vit. D | 11-15 | 1.5/1 | 11-15 | 1/1 | 11-15 | 1/1.5 |
| (>43) | 25(OH)D | 7.5-11 | (F.7) | 11-15 | (F.8) | 17-22.5 | (F.9) |

Remarks:
15 μg vitamin D per day (=600 IU) is according to the current view of diverse medical societies a normal, non-elevated daily supplementation dose!
In this table the calculated value of 11.25 has been rounded to 11. The findings summarized in Table 3 can be transformed into a flow chart which is provided herewith as FIG. 8. Based on this flow chart it has in a first step to be determined whether the obese individual belongs to the group of slightly, clearly or extremely obese subjects. In a second step it has to be determined whether the obese individual's blood level of 25(OH)D is or isn't reliably known. The following daily dosage regimens are then recommended on this basis:
(i) For slightly obese individuals of unknown or unreliably known level of 25(OH)D in blood, respectively, who have a normal lifestyle with respect to UV-light exposure and daily uptake of vitamin D, it is recommended to supplement vitamin D and 25(OH)D in a ratio of 1 to 1 and in amounts at the lower end of the range of 5 to 7.5 μg of each of the two products.
(ii) For slightly obese individuals of unknown or unreliably known level of 25(OH)D in blood, respectively, who have an unfavorable lifestyle with respect to UV-light exposure and daily uptake of vitamin D, it is recommended to supplement vitamin D and 25(OH)D in a ratio of 1 to 1 and in amounts at the higher end of the range of 5 to 7.5 μg of each of the two products.
(iii) For slightly obese individuals of reliably known and not much too low level of 25(OH)D in blood (>20 to 30 ng/mL) it is recommended to supplement vitamin D and 25(OH)D in a ratio of 1 to 1 and in amounts in the range of 5 to 7.5 μg of each of the two products.
(iv) For slightly obese individuals of reliably known and clearly low level of 25(OH)D in blood (>10 to 20 ng/mL) it is recommended to supplement vitamin D and 25(OH)D in a ratio of 1 to 1.5, keeping the dose of vitamin D at 5 to 7.5 μg and raising the dose of 25(OH)D to 7.5 to 11 μg.
(v) For slightly obese individuals of reliably known and very low level of 25(OH)D in blood (≤10 ng/mL) it is recommended to supplement vitamin D and 25(OH)D in a ratio of 1 to 2, keeping the dose of vitamin D at 5 to 7.5 μg and raising the dose of 25(OH)D to 10 to 15 μg.
(vi) For clearly obese individuals of unknown or unreliably known level of 25(OH)D in the blood, respectively, who have a normal lifestyle with respect to UV-light exposure and daily uptake of vitamin D, it is recommended to supplement vitamin D and 25(OH)D in a ratio of 1 to 1 and in amounts at the lower end of the range of 7.5 to 11 μg of each of the two products.
(vii) For clearly obese individuals of unknown or unreliably known level of 25(OH)D in the blood, respectively, who have an unfavorable lifestyle with respect to UV-light exposure and daily uptake of vitamin D, it is recommended to supplement vitamin D and 25(OH)D in a ratio of 1 to 1 and in amounts at the higher end of the range of 7.5 to 11 μg of each of the two products.
(viii) For clearly obese individuals of reliably known and not much too low level of 25(OH)D in blood (>20 to 30 ng/mL) it is recommended to supplement vitamin D and 25(OH)D in a ratio of 1.5 to 1, keeping the dose of vitamin D at 7.5 to 11 μg, which results in a dose of 25(OH)D of 5 to 7.5 μg.
(ix) For clearly obese individuals of reliably known and clearly low level of 25(OH)D in blood (>10 to 20 ng/mL) it is recommended to supplement vitamin D and 25(OH)D in a ratio of 1 to 1 and in amounts of 7.5 to 11 μg of each of the two products.
(x) For clearly obese individuals of reliably known and very low level of 25(OH)D in blood (≤10 ng/mL) it is recommended to supplement vitamin D and 25(OH)D in a ratio of 1 to 1.5, keeping the dose of vitamin D at 7.5 to 11 μg, which results in a dose of 25(OH)D of 11 to 17 μg.
(xi) For extremely obese individuals of unknown or unreliably known level of 25(OH)D in blood, respectively, who have a normal lifestyle with respect to UV-light exposure and daily uptake of vitamin D, it is recommended to supplement vitamin D and 25(OH)D in a ratio of 1 to 1 and in amounts at the lower end of the range of 11 to 15 μg of each of the two products.
(xii) For extremely obese individuals of unknown or unreliably known level of 25(OH)D in blood, respectively, who have an unfavorable lifestyle with respect to UV-light exposure and daily uptake of vitamin D, it is recommended to supplement vitamin D and 25(OH)D in a ratio of 1 to 1 and in amounts at the higher end of the range of 11 to 15 μg of each of the two products.
(xiii) For extremely obese individuals of reliably known and not much too low level of 25(OH)D in blood (>20 to 30 ng/mL) it is recommended to supplement vitamin D and 25(OH)D in a ratio of 1.5 to 1, keeping the dose of vitamin D at 11 to 15 μg, which results in a dose of 25(OH)D of 7.5 to 10 μg.
(xiv) For extremely obese individuals of reliably known and clearly low level of 25(OH)D in blood (>10 to 20 ng/mL) it is recommended to supplement vitamin D and 25(OH)D in a ratio of 1 to 1 and in amounts of 11 to 15 μg of each of the two products.
(xv) For extremely obese individuals of reliably known and very low level of 25(OH)D in blood (≤10 ng/mL) it is recommended to supplement vitamin D and 25(OH)D in a ratio of 1 to 1.5, keeping the dose of vitamin D at 11 to 15 μg, which results in a dose of 25(OH)D of 17 to 22.5 μg.

While there are indications in the flow chart (FIG. 8) and the above compilation for the cases of unknown or unreliably known blood levels of 25(OH)D, which address the use of dosages either approaching the lower or approaching the higher end of the ranges as shown in Table 3, respectively, there are no such indications for the cases of reliably determined corresponding blood levels. This does not mean that there are no criteria favoring the low versus the high end or the opposite. At the contrary, the measurement introduces an element to be considered in addition to the generalized lifestyle, namely the exact analytical value. This value may be at the higher or the lower end of the respective range of blood levels, and the two observables, the generalized lifestyle and the position of the blood level within the given range may both stipulate the same or stipulate opposite adaptations of the dose, respectively. A decision tree including all those different cases would become confusing. This has been the first of the inventors' two main reasons to keep the decision tree as simple as possible. The second has been their expectation that a skilled person will be able to interpret the principles upon which Table 3 and the decision tree are built and be able to take the right steps when determining the supplementation for a person formally falling into a given field of Table 3, but combining all kinds of conceivable extremes. Thus, a skilled person would, e.g., stick to the ratio 1/1, but even raise the dose beyond 11 μg per substance, if dealing with a person formally falling into field F.5 of Table 3, but combining 25(OH)D=10.5 ng/mL, BMI=43, total lack of sportiness and outdoor activities, age=60, height=2 m, and an unfavorable diet. Analogously, the skilled person would again stick to the ratio 1/1, but lower the dose even below 7.5 μg per substance, for a person formally falling into the same field F.5 of Table 3, but combining the opposite extremes, viz. blood level of 25(OH)D=19.5 ng/mL, BMI=37, distinct sportiness and a lot of outdoor activities, age=25, height=1.5 m, and a favorable diet. In other words: The skilled person is expected to, e.g., realize that the amount of non-fatty tissues and blood, main target compartments of 25(OH)D, is in comparison to an obese and very tall person significantly lower in a very short person with the same BMI. Finally, obese individuals with a blood level of 25(OH)D>30 ng/mL do not need vitamin D supplementation, unless particular circumstances which are not or not directly related to their obesity, respectively, ask for it.

The term “lower end of the range” as used herein is meant to encompass the range from the lowest end of the range up to the middle of the specified range.

The term “higher end of the range” as used herein is meant to encompass the range from the middle of the specified range up to the highest end of the range.

The term “normal lifestyle with respect to UV-light exposure” is meant to be a qualified estimation by a skilled person of the average amount of hours per day a patient is exposed to UV-radiation from natural sources (sunlight) and/or from UV-radiation lamps during a given period of time such as e.g. the last several weeks or the last few months. The UV light exposure can also be derived based on measurements by a small wearable device such as the one described in an article in ScienceDaily on Jan. 9, 2018 (see: Northwestern University. “World's smallest wearable device warns of UV exposure, enables precision phototherapy.” ScienceDaily. ScienceDaily, 6 Dec. 2018. <www.sciencedaly.com/releases/2018/12/181206114707.htm; last asessed Mar. 21, 2021).

As understood herein, 25(OH)D3 dosage may undergo adaptations based on analytical blood level determinations. The fields highlighted in bold in Table 3 address cases of obesity plus insufficiency/deficiency most likely encountered in modern civilizations. According to the present invention, the respective dosages and ratios may in healthy obese adults be applied without frequent measurements of the vitamin D status. According to the present invention, persons who are tall and obese (or heavy and obese) should preferably be dosed according to or go for the higher end, respectively, of the applicable ranges given in the table. The same holds for people at the more unfavorable end of the indicated ranges of insufficiency or deficiency, respectively. The lower end of the range in the table's upper left field is preferably also suitable for individuals who are non-obese, but overweight and borderline insufficient as well as individuals just having a tendency to get obese (e.g. based on genetics).

For the individuals with the highest BMI, the 25(OH)D3 dosages in the above Table 3 are cautiously increased by raising the dose and leaving the ratio unchanged, to ensure adequate, but non-exaggerated supplementation of adipocytes with vitamin D per se and to normalize the (blood level-based) vitamin D status with 25(OH)D. Considering that our experimental results showed no statistically significant BMI-dependence of the improvement as achieved by a given dose of 25(OH)D3, one might have expected no raising of its dose. There are several reasons for raising it anyway: We cannot absolutely exclude a slight trend towards lower levels achieved when dosing 25(OH)D to obese subjects. The total blood volume (BV) of a person of a given height is increasing with increasing BMI—proportional to its square root. Finally, there likely is a feedback control mechanism mitigating the release from adipocytes of vitamin D (and/or vitamin D metabolites), if the blood level of the metabolites gets higher.

Such a feedback control may be regarded as logical addition to the often-cited evolutionary role of the deposition of vitamin D in fatty tissues. It would have kept vitamin D3 levels in adipocytes high in summer when 25(OH)D3 blood levels were high and growth of fatty tissue was favorable, and it would have fostered vitamin D release and the burning of body fat when the sun was low and little vitamin D3 was formed. An argument in favor of the mentioned feedback control is, by the way, the less than additive increase of the 25(OH)D3 level caused by parallel administration of 25(OH)D3 and vitamin D3 (cp. the already mentioned article by Alexander Jetter et al., Bone 59, 14-19 (2014)). The main differences between the present invention and the study of Jetter et al. are that Jetter et al. does not address obesity and uses doses which differed from ours. Jetter's study supports the interpretation that the two, D3 and 25(OH)D3 go to different compartments without mutually influencing the initial distribution to a significant extent. The study can be interpreted in the sense that the clear effect upon the vitamin D status 25(OH)D3 has, is marginally affected, if at all, by the parallel administration of D3 and that there seemingly is a trend towards further weakening of the anyway weak effect D3 has upon the vitamin D status (viz. the 25(OH)D3 blood level), if it is dosed in parallel to 25(OH)D3.

Pointing in the same direction is the observation that a given dose of vitamin D raises a comparatively high baseline level of 25(OH)D to a weaker absolute extent than it raises a comparatively low baseline level of 25(OH)D (cp. Muhammad M. Hammami et al., BMC Endocr. Disord. 2017; 17: 12—https.//www.ncbi.nlm.nih.gov/pmc/articles/PMC5324269/). Assuming that this discussed feedback control exists leads to the conclusion that a relatively low (blood level-based) vitamin D status may be desirable, if the focus is on controlling obesity. However, the approach has to be kept within reasonable limits. Thus, more than 30 ng/mL of 25(OH)D may be targeted in cases of extreme obesity (which probably were never encountered in ancient times), in order to avoid unhealthy vitamin D depletion of adipocytes. In cases of moderate and healthy obesity, however, 30 ng/mL but not more may be regarded as reasonable compromise, unless there are other more relevant priorities, like, e.g., the stimulation of the immune system to mitigate the effect of viral infections.

The proposed vitamin D supplementation scheme of the present invention, according to Table 3, shall now be discussed in view of the whole population's distribution of heights and weights as visualized in Table 1 for American male adults. Looking at the extremes Table 1 covers (upper left and lower right fields) leads to factors of approximatively (≈) 1.33 for heights, ≈2.25 for BMIs, and ≈4 for weights. Using the respective cited formula further leads to factors of ≈11.8 for total body fat weight and (assuming congruence)≈5.2 for the body fat surface, which as discussed herein is expected to govern at least short-term deposition. As non-congruency will rather reduce than add to the surface factor, as deposition increases with increasing fatty tissue surface, and as a BMI of 20 does not have to be considered in the context of obesity, it can be estimated that the factor 3 separating the lowest and the highest vitamin D3 dose according to Table 3 is roughly what it takes to reasonably cover the adipocytes' respective needs in different healthy obese or potentially obese individuals.

The combination of 25(OH)D3 and vitamin D3 is the prototype of the present invention. However, as understood herein, it can employ the vitamin D per se and the 25-hydroxylated metabolite, respectively, it can be based on the D3- and/or the D2-series, and it can be based on all the conceivable combinations of those options. The considerations of ratio of 25(OH)D/vitamin D in this combination and the dosages can in terms of the basic concept presented be extrapolated to the other combinations of two corresponding components and to all the combinations of three or four components. The reason is the strong similarity of the two vitamins, on the one hand, and the analogous similarity of the two 25-hydroxylated metabolites, on the other hand. Of particular interest amongst the two component combinations is the one of 25(OH)D2+vitamin D2. It consists exclusively in plant-derived or fully synthetic materials. Correspondingly, it is or can be obtained in strictly vegan quality, respectively.

The vitamin D3-series is as known to the skilled person more metabolically active overall in terms of impacting PTH levels and possibly in terms of the number of genes impacted with regard to their expression by a given dose in vitro. One reason is that the structural difference in the sidechain impacts the activating 25- and the inactivating 24-hydroxylation. Furthermore, the DBP-complex is less stable in the D2-series, the induction of the 24-hydroxylating CYP24A1 is stronger in the D3-series and there is a statistically highly significant difference in timing as follows (cp. the already cited publication by Muhammad M. Hammami et al., BMC Endocr. Disord. 2017; 17: 12): Vitamin D2 results in a higher overall area under the curve (AUC) of 25(OH)D, if administered daily over many weeks, rather than 2- or 4-weekly as bolus, while the opposite holds for vitamin D3. This points to comparatively more accelerated deposition at elevated concentration in case of vitamin D2. It may in addition point to a less distinct feedback-suppression by elevated 25(OH)D blood levels of the release of adipocyte-bound vitamin D3 than vitamin D2.

Finally, it is reasonable to assume that there might be some differences of the two vitamins regarding their distribution in the organism. The combination of vitamin D2 and D3 shows less toxicity than straight additivity of the toxic effects of the two would predict. Dosing 25(OH)D plus the combined vitamins D2 and D3 to obese individuals will at comparatively low dose levels result in adequate supply of the required amounts of vitamin D to their different types of adipocytes and mitigate local overdosing. Dosing 25(OH)D plus vitamin D3 represents the supplementation approach of choice for overweight or obese individuals in environments of significant vitamin D2 fortification of food.

It is further noted that 25(OH)D3 and 25(OH)D2 plus vitamin D in combination may be more suitable than just one 25-hydroxylated metabolite plus vitamin D in view of the compromise to reach a reasonable vitamin D status and still enable some level of release of vitamin D from adipocytes.

The inventors compiled some essentially indirect evidence up to today for their in view of the state-of-the-art surprising conclusion that the relevant deposition of vitamin D in fatty tissues including lipid droplets is tissue-surface-based rather than tissue-mass-based. In addition, they pursue experiments targeting a direct proof of their conclusion, which may be compared with the above-mentioned observations concerning free cholesterol (already cited article by Susanne Prattes et al.). But they are not yet there, and the surface-based deposition is still to an extent a hypothesis. However, the inventors found that the experience that obese individuals need about 2.5 times the amount of vitamin D supplementation a normal individual needs to achieve a given level of 25(OH)D provides an independent basis to support their surprising supplementation- and dosage-related conclusions—and the dosage figures resulting are nicely comparable. Thus, it is proposed that the reasonable ratio of 25(OH)D to vitamin D to be preferentially supplemented for obese can be determined as follows. If one estimates that ⅔ of vitamin D administered to non-obese quickly finds its way from the lymph to the blood and that the rest ends up in adipose tissue (disregarding other compartments) and assuming that 100% of the administered vitamin D are actually absorbed (which is on the high side) and assuming that 600 I.U. per day is for sure a healthy supplementation, one can conclude that about 200 I.U. per day are going to the adipose tissue of non-obese. If one now supplements the 2.5-fold amount, viz. 1500 I.U. per day to an obese person, then 400 I.U. (the same amount as in normal subjects) will end up in the blood and will eventually be metabolized to 25(OH)D. But that means on the other hand that 1100 I.U. vitamin D per day is loaded into the adipose tissue of the obese person. In other words: about 66% of the administered vitamin D are eventually becoming 25(OH)D in the blood of non-obese, while only 26.66% of the administered vitamin D become 25(OH)D in the blood of obese. Therefore, based on the rule of thumb that obese persons need 2.5-times more vitamin D to get to the same adequate vitamin D-status (measured as 25(OH)D-level in blood) and assuming as above that 100% are absorbed from the gastrointestinal tract (GI-tract) then 5.5—times more vitamin D is ending up in the adipose tissue of an obese person compared to a non-obese person.

If finally assuming that zero vitamin D per day in the adipose tissue might be too little (in other words, that one should not exclusively supply 25(OH)D!), one might conclude that 200-400 I.U. vitamin D ending up per day in the adipose tissue of obese are fine or possibly even advantageous (200 I.U. were calculated above on the basis of a supplementation of 600 I.U. to a non-obese, and 400 I.U. might analogously still be fine in an obese who may, in comparison to the non-obese, have roughly the double amount of fatty tissue, and roughly the 1.5 times larger surface of fatty tissue). Thus, one might supplement obese by administering up to about 550 I.U. vitamin D per day (if 26.66% thereof are eventually becoming 25(OH)D, 73.33% are ending up in fatty tissue; and 73.33% of 550 I.U. are 400 I.U.—or precisely 403.15) and correct the resulting deficient blood level by dosing 25(OH)D in parallel, e.g. as well up to about 13.75 μg (viz. 1 μg vitamin D=40 I.U.) to make up for the fact that the absorption may not be complete. The administration of 550 I.U. of vitamin D results, based on the rule of thumb, in 26.66% in the blood of obese, corresponding to the formation of approx. 3.7 μg 25(OH)D; therefore, we lack approx. 6.3 μg 25(OH)D, which have to be supplemented as such. If we now supplement 13.75 μg 25(OH)D instead, we take into account that the enterohepatic up-take is probably lower than 100%, that approximately doubling that dose keeps us still very far away from a toxic dose, and that the obese might in addition be a bit heavier than the non-obese and accordingly have a statistically somewhat higher blood-volume. Overall, we stay on the careful side for the obese with regard to the case of vitamin D, where we do not want to overshoot and where we assume 100% up-take from the GI-tract, and we are somewhat more aggressive in the case of 25(OH)D, because we know that it has a nice therapeutic width and that it is not accumulating in fatty tissues.

Based on the above it is suggested that a combination of 25(OH)D2 and vitamin D3 or vitamin D2 or a combination of 25(OH)D3 and vitamin D3 or vitamin D2 is used for the treatment of and/or prophylaxis of vitamin D insufficiency or deficiency in obese persons, persons having the tendency to get obese or persons having a history of malabsorption of vitamin D. The ratio of the 25 hydroxy form of the vitamin D to vitamin D is in the range between 1 to 5 and 5 to 1, preferentially between 1 to 2 and 2 to 1, or in a ratio of 1 to 1, most preferably in a ratio selected from the group of 1 to 1, 1 to 1.5, 1 to 2, 1.5 to 1. Under certain circumstances (e.g. depending on the lifestyle—such as when a person has a high exposure of his skin to the sun) the supplementation of 25(OH)D2 and/or 25(OH)D3 may already be sufficient due to the endogeneous vitamin D3 production.

### Example 1

The conclusions as presented herein are based on the following experiments: In a double-blinded cross-over study single high dosages (900 μg total in two soft gel capsules containing 450 μg) of either vitamin D3 or 25(OH)D3 were administered to vitamin D-deficient volunteers including healthy controls as well as subjects with a history of malabsorption. At the outset, the BMI was determined. Blood samples were then taken at baseline and at 2, 4, 6, 8, and 12 hours as well as 1, 2, 3, 7, and 14 days after administration (FIG. 2). All the samples were analyzed for their contents in vitamin D3 per se as well as 25(OH)D3. It was found that the subjects with a history of vitamin D/fat malabsorption showed after administration of the respective product reduced vitamin D3, but almost normal 25(OH)D3-bioavailability (FIGS. 3a and b). Moreover, it was found in particular that the subjects with an elevated BMI compared as follows to normal subjects (with a BMI <30): The bioavailability (peak level and area under the curve) after administration of vitamin D3 was again comparatively strongly reduced, while the respective values obtained after administration of 25(OH)D3 were essentially the same. FIG. 4 visualizes these results. In addition, peak levels were more rapidly reached with 25(OH)D3 than with vitamin D3 (see FIG. 5). The immediate increase in serum 25(OH)D3 compared to the delayed increase observed with vitamin D3 supports the concept that 25(OH)D3 is being rapidly absorbed from the small intestine into the portal circulation, whereas vitamin D3 first needs to be incorporated into the chylomicrons which are absorbed into the lymphatic system. The lymphatic system then drains into the superior vena cava where vitamin D3 finally enters the circulation.

The observed difference reflects the published (Hossein-nezhad A.; Mayo Clin. Proc. (July 2013); cited above) difference of the two substance types' main mechanisms of up-take from the gastro-intestinal tract: mainly lymphatic (in chylomicrons) in case of vitamin D and mainly enterohepatic (via portal vein) in case of 25(OH)D. But those different mechanisms of up-take do per se not explain the strong BMI-dependence of the appearance in blood of vitamin D and correspondingly its metabolite, 25(OH)D, after single doses of vitamin D as observed in our study: rapid and very distinct in case of BMI <20, less rapid and distinct in case of BMI greater than 20 up to a BMI about 30, and somewhat slower and strongly reduced in case of BMI higher than 30 (DiVasta A D, et al. J Clin Endocrinol Metab (August 2011); cited above).

There are several conceivable theoretical approaches to explaining this BMI-dependence. There are indications for a strongly uneven distribution to different fatty compartments of administered vitamin D and indications for a slower rate of release from these compartments into the bloodstream in obese as opposed to non-obese. Taken together, our observations and considerations lead to the following conclusions:
a) It is too simplistic to just explain the reduced appearance of administered vitamin D in the blood of obese as compared to non-obese by referring to the obese subjects' comparatively higher amounts of total fatty tissue. Surface-dependent deposition, a certain feedback control by vitamin D metabolites of the release of vitamin D from adipocytes (possibly in part in metabolized form), and a role of the differences of the lymphatic systems of obese and non-obese persons are likely to contribute herein.
b) It is very likely that significant accumulation and strongly elevated local concentrations of vitamin D in given populations of adipocytes or adipose tissues result, if administering to obese 2.5 to 3 or even more times the dose normally administered to non-obese.
c) It is conceivable that this accumulation is in obese or potentially obese subjects (due to local metabolism) resulting in counterproductive or negative local effects, while there may still be absolutely no signs of systemic hypercalcemia because of the substances' very slow release from the respective sites (body fat pool).

As noted above vitamin D formulations for use in the treatment of persons with vitamin D deficiencies are commercially available for decades. Pharmaceutical compositions comprising 25(OH)D3 or 25(OH)D2 can be formulated by the skilled person in a similar way taking into account the information on the properties, specifications and characteristics of suitable excipients as described e.g. in standard texts such as Fiedler, H. P.; 1996; Lexikon der Hilfsstoffe for Pharmazie, Kosmetik und angrenzende Gebiete; Editio Cantor Verlag Aulendorf (Germany), and Kibbe, A. H.; 2000; Handbook of Pharmaceutical Excipients, a joint publication of Pharmaceutical Press, London (UK), and American Pharmaceutical Association, Washington (US) as well as manufacturers' brochures.

Immediate-release (IR) formulations of calcifediol (25(OH)D3) have been available for decades in the European Union (EU) for indications such as rickets, prevention of calcium disorders secondary to corticosteroid or anticonvulsant therapy and treatment of osteomalacia, renal osteodystrophy, hypoparathyroidism, familial hypo-phosphatemia and vitamin D malabsorption (see: Holick M F Binkley N C, Bischoff-Ferrari H A, Gordon C M, Hanley D A, Heaney R P, Murad M H and Weaver C M. “Evaluation, Treatment & Prevention of Vitamin D Deficiency: An Endocrine Society Clinical Practice Guideline”; J Clin Endocrinol Metab. (July 2011) 96(7):1911-1930; Kidney Disease: Improving Global Outcomes (KDIGO) CKD-MBD Update Work Group. KDIGO 2017 Clinical Practice Guideline Update for the Diagnosis, Evaluation, Prevention, and Treatment of Chronic Kidney Disease-Mineral and Bone Disorder (CKD-MBD). Kidney Int Suppl. 2017; 7: 1-59).; Haddad J G, Jr. Rojanasathit S.; “Acute administration of 25-hydroxycholecalciferol in man” J. Clin Endocrinol Metabo (1976) 42:284-290; Bordier P J, Marie P J, Arnaud C D; “Evolution of renal osteodystrophy: correlation of bone histomorphometry and serum mineral and immune parathyroid hormone values before and after treatment with calcium carbonate or 25-hydroxycholecalciferol”; Kidney Int Suppl. (January 1975) 102-112).

In the US, IR calcifediol was marketed from 1980 to 2002 as Calderol™ for the treatment of metabolic bone disease in dialysis patients and was withdrawn from the market in 2002 for commercial reasons not associated with safety or efficacy. Calderol™ displayed pharmacokinetic (PK) characteristics consistent with an IR formulation, with the time (tmax) to reach maximum serum calcifediol concentrations (Cmax) occurring within 4 to 8 hours postdose (Haddad J G, Jr.; cited above). Published clinical studies have clearly shown that IR calcifediol increased serum 25(OH)D far more quickly and effectively than vitamin D supplements. Extended release calcifediol gradually releases calcifediol, increasing serum 25(OH)D at a slower rate than IR formulations (Sprague et al. 2017; cited above; Sprague et al. 2015; cited above).

Pharmaceutical compositions comprising a combination of 25(OH)D3 and 25(OH)D2; 25(OH)D3 and vitamin D3 or vitamin D2; or 25(OH)D2 and vitamin D3 or vitamin D2; or a combination of 25(OH)D3 or 25(OH)D2 and vitamin D3 and/or vitamin D2 can be prepared by the skilled person in similar way as described above.

The pharmaceutical compositions as described above may contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

### Example 2

The pharmacokinetic parameters of orally administered 25(OH)D3 and vitamin D3 (both provided for formulation in form of visually identical soft-gel pills) were studied based on the corresponding serum concentration-time curves in healthy adults and adults with a history of intestinal malabsorption (see FIG. 2). Cinical safety has been evaluated by monitoring serum calcium, phosphorus, parathyroid hormone (PTH) and 25(OH)D levels at the beginning and end of the study. Furthermore, the distribution of supplemented 25(OH)D3 (or 25(OH)D2) into adipose tissue and the role it is playing there was also studied.

Study outcomes measured are vitamin D and its major metabolite 25(OH)D, whereby the measurements were taken at various times over a 2-week period. Serum levels of calcium, phosphorus, albumin, creatinine and intact parathyroid hormone (iPTH) were determined at baseline and at the end of each 14-day pharmacokinetic study (see Table 4). The pharmacokinetic parameters have been determined by evaluating the serum concentration-time curves in patients with malabsorption syndrome and healthy controls (Tables 5 and 6).

The primary outcomes of the study are the serum pharmacokinetic parameters of 25(OH)D3 and vitamin D3 after a single oral administration of 900 μg of each in a double-blind randomized crossover study. The serum concentrations of vitamin D and 25(OH)D at various times were determined according to methods known in the art as the primary outcome measure.

The study (summarized in FIG. 2) was designed as follows:

Adult patients (18 years and older) with a history of intestinal malabsorption (n=10) and healthy adult volunteers (n=10); (of both sexes and all ethnicities) has been recruited in this randomized double-blinded crossover study. The BMI has been determined and recorded at baseline. All subjects have been prescreened for 25(OH)D levels to include subjects with 25(OH)D<30 ng/mL. The subjects have been randomized to receive two oral doses of 450 μg taken simultaneously (total 900 μg) of either vitamin D3 or 25(OH)D3. Blood samples were taken at baseline and at 2, 4, 6, 8, 12 hours and days 1, 2, 3, 7 and 14. There was a two weeks washout period before the subject received in a double blinded manner, two oral doses of 450 μg taken simultaneously (total 900 μg) of either 25(OH)D3 or vitamin D3 (depending on which one they took in the randomization) any time after the 2 week washout. Blood samples were taken at baseline and at 2, 4, 6, 8, 12 hours and days 1, 2, 3, 7 and 14 days. Serum levels of calcium, phosphorus, albumin, creatinine, and intact parathyroid hormone (iPTH) were obtained at baseline and at day 14 for each arm (or within a reasonable time if the study participant was unable to come on the exact day). Vitamin D and 25(OH)D were determined on all the blood samples collected at the various times.

The clinical study was performed taking into account the following potential risks and benefits. Pharmacokinetic studies have previously been performed with 25(OH)D3 given as a single 448 μg dose intravenously or as a single dose of 900 μg in a slow release formulation (Sprague et al. 2017; cited above). As can be seen in FIG. 3 of Sprague et al., the peak concentration of 25(OH)D increased by approximately 110 ng/mL and 10 ng/mL above the baseline after receiving the bolus intravenous dose of 448 μg or 900 μg of slow release 25(OH)D3, respectively. No toxicity was observed.

In the course of the present clinical study only those subjects were included who had a level of 25(OH)D<30 ng/mL. Therefore, it was concluded that an increase of as much as 110 ng/mL after a bolus intravenous dose which by 24 hours decreased to 40 ng/mL will not cause toxicity. Vitamin D intoxication is seen only when the 25(OH)D levels are sustained above 150 ng/mL for a prolonged period viz. usually several months. A single oral dose of 1250 μg of vitamin D once a week for 8 weeks or every 2 weeks for up to 6 years is not associated with toxicity (see: Holick M F et al.; J Clin Endocrinol Metab. (July 2011); cited above).

Safety issues are associated with the potential for vitamin D intoxication. Vitamin D intoxication is associated with hypercalcemia, hyperphosphatemia and suppressed PTH as noted by the Endocrine Society Practice Guidelines on Vitamin D (see: Holick M F et al.; J Clin Endocrinol Metab. (July 2011); cited above; and Kidney Disease Improving Global Outcomes (KDIGO) 2017 clinical practice guideline update for the diagnosis, evaluation, prevention and treatment of chronic kidney disease—mineral and bone disorder (CKD-MBD). Kidney Int Suppl. 2017; 7: S1-S59). As all subjects involved in the study are vitamin D deficient or have insufficient levels of vitamin D (25(OH)D<30 ng/mL), it was presumed that they are not harmed by receiving single doses of both vitamin D3 and 25(OH)D3, because such treatment will rather help to improve their vitamin D status. Men and women who took 50,000 IU (1250 μg) vitamin D once a week for 8 weeks were able to maintain their serum 25(OH)D at >30 ng/mL without toxicity (Holick M F et al. (July 2011); cited above).

Study Subject Selection

Subject Inclusion Criteria
1. 18 years of age or older
2. Healthy male or female adults (for healthy control group)
3. Adult male or female patients with a history of intestinal malabsorption at the Boston University Medical Campus.
4. No medications or disorders that would affect vitamin D metabolism.
5. Taking vitamin D (ergocalciferol or cholecalciferol). Subjects taking vitamin D supplement of more than 2000 IUs (50 micrograms/day) that may interfere with study endpoints, must be willing and able to discontinue use of these supplements for the duration of the study and allow for at least a 14-day washout prior to enrollment.
6. Women must be on birth control and not pregnant based on a negative pregnancy test at baseline for each of the two pharmacokinetic studies.
7. Ability and willingness to give informed consent and comply with protocol requirements.
8. Serum total 25(OH)D<30 ng/mL.

Subject Exclusion Criteria
1. Use of supplemental dose of vitamin D2 (ergocalciferol) or vitamin D3 (cholecalciferol) daily equivalent (2,000 IU/50 μg) 1 week or less prior to randomization and during the study.
2. On medications that can affect vitamin D metabolism including steroids such as prednisone, anti-seizure medications and medications to treat HIV.
3. Tanning in a tanning bed at least one week before the study and throughout the duration of this study.
4. Anyone anticipating going on holiday (where exposure to sun is imminent) 1 week before or during the entire study.
5. Participation in the study or any reason which, in the opinion of the investigator, makes adherence to a treatment or follow-up schedule unlikely.
6. History of an elevated serum calcium.
7. Chronic hepatic or renal failure.
8. Subjects with a history of an adverse reaction to orally administered vitamin D.
9. Inability to give informed consent.

The subjects baseline characteristics are provided in the following Table 4.

TABLE 4
Baseline characteristics of healthy participants
and patients with fat malabsorption

| | | Patients | |
|---|---|---|---|
| | Healthy | with fat | |
| | participants | malabsorption | p- |
| | (N = 10) | (N = 6) | value |
| Age | 32.3 ± 2.7 | 46.5 ± 4.1 | 0.011* |
| Number of female participants | 8 (80%) | 6 (100%) | 0.500 |
| Body mass index (kg/m²) | 27.0 ± 2.1 | 32.7 ± 4.1 | 0.263 |
| Ethnicity | | | |
| Caucasian | 5 (50%) | 4 (67%) | |
| Hispanic | 0 (0%) | 1 (17%) | |
| Asian | 2 (20%) | 0 (0%) | |
| Black | 3 (30%) | 1 (17%) | |
| Serum chemistry | | | |
| Vitamin D2 (ng/mL) | 0.0 ± 0.0 | 0.0 ± 0.0 | 0.100 |
| Vitamin D3 (ng/mL) | 0.0 ± 0.0 | 1.6 ± 1.0 | 0.313 |
| Total 25-hydroxyvitamin | 17.1 ± 2.3 | 14.7 ± 3.4 | 0.958 |
| D (ng/mL) | | | |
| 25-hydroxyvitamin D2 (ng/mL) | 0.4 ± 0.4 | 4.2 ± 3.1 | 0.428 |
| 25-hydroxyvitamin D3 (ng/mL) | 16.7 ± 2.1 | 10.5 ± 4.2 | 0.368 |
| Intact PTH (pg/mL) | 41.5 ± 5.4 | 74.0 ± 16.9 | 0.073 |
| Total calcium (mg/dL) | 9.4 ± 0.1 | 9.4 ± 0.1 | 0.875 |
| Phosphate (mg/dL) | 3.9 ± 0.3 | 4.0 ± 0.3 | 0.635 |
| Creatinine (mg/dL) | 0.8 ± 0.03 | 0.7 ± 0.04 | 0.147 |
| eGFR (mL/min/1.73 m²) | 106.8 ± 4.7 | 104.0 ± 6.7 | 0.792 |
| Glucose (mg/dL) | 83.1 ± 7.5 | 89.8 ± 8.8 | 0.263 |
| Albumin (g/dL) | 4.4 ± 0.08 | 4.1 ± 0.05 | 0.022* |
| Total protein (g/dL) | 6.9 ± 0.08 | 6.8 ± 0.2 | 0.635 |
| Total bilirubin (mg/dL) | 0.5 ± 0.09 | 0.5 ± 0.05 | 0.792 |
| Alkaline phosphatase (U/L) | 54.5 ± 4.8 | 88.5 ± 17.3 | 0.022* |
| Aspartate | 18.4 ± 1.6 | 18.7 ± 2.5 | 0.875 |
| aminotransferase (U/L) | | | |
| Alanine aminotransferase (U/L) | 15.6 ± 1.0 | 16.7 ± 4.1 | 0.958 |

Data are expressed as mean ± SEM or number of participants with %.
*denotes statistically significant difference between groups (p < 0.05).

The individuals participating in the study had, after signing a consent form, been randomized by a computer randomization chart in a blinded manner to receive two oral doses of 450 μg taken simultaneously (total 900 μg) of either vitamin D3 or 25(OH)D3. Blood samples have been taken at baseline and at 2, 4, 6, 8, 12 hours and days 1, 2, 3, 7 and 14. After a washout period of at least 14 days (2 weeks) the subjects received in a double blinded manner two oral doses of 450 μg taken simultaneously (total 900 μg) of either 25(OH)D3 or vitamin D3 (depending on which one they took in the first randomization) any time after the washout. Blood samples were taken at baseline and at 2, 4, 6, 8, 12 hours and days 1, 2, 3, 7 and 14.

All subjects have been recruited either from the Boston University Medical Campus or from the Boston area using a flyer that was be posted for recruitment purposes. In collaboration with the Endocrine, Diabetes and Nutrition and Gastroenterology sections in the Department of Medicine and the Surgical Department of Boston University Medical Campus patients with a malabsorption syndrome were invited to participate in the study. All subjects have been prescreened for serum 25(OH)D levels and included if they were vitamin D deficient or insufficient viz. a 25(OH)D<30 ng/mL. (Holick et al JCEM 2011.; cited above).

Blinding of capsules was done by computer randomization and provided by the Investigational Pharmacy Service during the trial. All subjects and research staff involved in the clinical study were blinded during the clinical trial. The data were unblinded at the end of the data collection phase at the discretion of the Principal Investigator. There were no unforeseen medical circumstances which required the premature unblinding of participant data under any of the participant's health Assessment of Safety and Data Safety Monitoring Plan (DSMP). The major potential side effect is hypercalcemia, hyperphosphatemia and suppressed PTH level with a 25(OH)D>150 ng/mL. However, toxicity has never been reported after a single or multiple oral doses of 1250 μg vitamin D or after a dose of 900 μg 25(OH)D3.

For the pharmacokinetic evaluation the sample size for this pilot study was estimated to be 10 in each group (10 patients with malabsorption syndrome and 10 healthy control) based on Rochon's sample size computation method for repeated measurement experiments (for details see: Kloprogge F, Simpson J A, Day N P J, White N J, Tarning J. 1975; 102-112. “Statistical power calculations for mixed pharmacokinetic study designs using a population approach” AAPS J. (September 2014) 16(5):1110-1118. doi: 10.1208/s12248-014-9641-4. Epub 2014 Jul. 11.-14); Kang D, Schwartz J B, Verotta D. “Sample size computations for PK/PD population models” J Pharmacokinet Pharmacodyn. (December 2005) 32(5-6):685-701; Aarons L, Ogungbenro K. “Optimal design of pharmacokinetic studies” Basic Clin Pharmacol Toxicol. (March 2010); 106(3):250-255). Also considered were the data which were obtained from previous clinical trials for vitamin D3 and 25(OH)D3 (for details see: Lo C W et al.; cited above; Farraye F A, et al.; cited above; Sprague S M et al, 2017; cited above; Sprague S M, et al. 2015; cited above). This sample size of this study is expected to provide the power of >0.80.

Differences between treatment groups were analyzed by a one- or two-sided t-test, as appropriate, with statistical significance set at p<0.05. The pharmacokinetic (PK) parameters for baseline-corrected serum 25(OH)D and vitamin D will be derived using non-compartmental analysis. Area under the serum concentration-time curve (AUC) was estimated using the linear trapezoid rule. The t½ was calculated by determination of the elimination rate constant from concentration values in the follow-up period.

As expected, healthy adults were able to absorb both vitamin D3 and 25(OH)D3 as demonstrated by significant increases in the blood levels of vitamin D3 and 25(OH)D3 after receiving a single 900 μg oral dose of vitamin D3 and 25(OH)D3, respectively. (FIG. 3) Patients with documented fat malabsorption syndromes were not able to raise their blood levels of vitamin D3 after an oral dose of 900 μg of vitamin D3 to the same degree as healthy adults (FIG. 3). It was found, however that these same malabsorption patients were able to more efficiently absorb 25(OH)D3 after an oral dose of 900 μg of 25(OH)D3 (two 450 mcg capsules) like healthy adults (FIG. 3).

More surprising was, finally, the finding illustrated by Table 6 and by FIG. 4a and FIG. 4b, namely that the blood level of healthy persons with lower and higher BMI, respectively, did significantly differ when administering and analytically determining serum vitamin D3, but did not when administering and analytically determining serum 25(OH)D3. In the case of vitamin D3 the statistical procedures as described above delivered p-values of 0.016 and 0.010 for the differences regarding the AUC and T_{1/2}, respectively. In the case of administration of 25(OH)D3 to the identical persons, however, there were absolutely no such BMI-dependent differences (p-values between 0.42 and 1). This is remarkable, if considering the small size of the two groups (N=5) and supports our health-related conclusions.

TABLE 5
Pharmacokinetic parameters of oral 900 μg vitamin
D3 and oral 900 μg 25-hydroxyvitamin D3 in healthy
participants and patients with fat malabsorption

| | | Patients | |
|---|---|---|---|
| | Healthy | with fat | |
| | participants | malabsorption | p- |
| | (N = 10) | (N = 6) | value |
| 900 μg vitamin D3 arm | | | |
| AUC (ng · hr/mL) | 3258 ± 496 | 1177 ± 425 | 0.022* |
| Cₘₐₓ (ng/mL) | 53.5 ± 6.0 | 24.3 ± 8.4 | 0.016* |
| Tₘₐₓ (hr) | 10.4 ± 0.7 | 11.3 ± 0.7 | 0.345 |
| T_{1/2} (hr) | 31.4 ± 3.3 | 28.7 ± 1.5 | 0.713 |
| C_{trough} (ng/mL) | 0.3 ± 0.3 | 0.1 ± 0.1 | 0.220 |
| 900 μg 25-hydroxyvitamin | | | |
| D3 arm | | | |
| AUC (ng · hr/mL) | 3128 ± 545 | 2667 ± 735 | 0.562 |
| Cₘₐₓ (ng/mL) | 23.1 ± 4.6 | 23.2 ± 6.8 | 1.000 |
| Tₘₐₓ (hr) | 11.2 ± 4.1 | 5.3 ± 0.7 | 0.031* |
| T_{1/2} (hr) | 60.6 ± 7.9 | 65.7 ± 29.9 | 0.313 |
| C_{trough} (ng/mL) | 6.1 ± 1.3 | 6.7 ± 1.5 | 0.875 |

Data are expressed as mean ± SEM.
*denotes statistically significant difference between groups (p < 0.05).
Abbreviations: AUC: area under the concentration-time curve; Cₘₐₓ: maximal concentration; Tₘₐₓ: time to maximal concentration; T_{1/2}: elimination half-life; C_{trough}: trough level at day 14

TABLE 6
Pharmacokinetic parameters of oral 900 μg vitamin
D3 and oral 900 μg 25- hydroxyvitamin D3 in healthy
participants with higher and lower body mass index

| | Healthy | Healthy | |
|---|---|---|---|
| | participants | participants | |
| | with higher | with lower | |
| | BMI | BMI | p- |
| | (N = 5) | (N = 5) | value |
| 900 μg vitamin D3 arm | | | |
| AUC (ng · hr/mL) | 2089 ± 547 | 4427 ± 350 | 0.016* |
| Cₘₐₓ (ng/mL) | 44.4 ± 9.3 | 62.6 ± 5.7 | 0.310 |
| Tₘₐₓ (hr) | 9.6 ± 1.0 | 11.2 ± 0.8 | 0.310 |
| T_{1/2} (hr) | 23.8 ± 3.1 | 39.0 ± 3.4 | 0.010* |
| C_{trough} (ng/mL) | 0.0 ± 0.0 | 0.6 ± 0.6 | 0.690 |
| 900 μg 25-hydroxyvitamin D3 arm | | | |
| AUC (ng · hr/mL) | 2621 ± 855 | 3633 ± 690 | 0.421 |
| Cₘₐₓ (ng/mL) | 21.4 ± 8.0 | 24.8 ± 5.5 | 0.548 |
| Tₘₐₓ (hr) | 15.2 ± 8.2 | 7.2 ± 0.5 | 0.841 |
| T_{1/2} (hr) | 55.0 ± 10.7 | 66.2 ± 12.3 | 1.000 |
| C_{trough} (ng/mL) | 4.6 ± 1.4 | 7.6 ± 2.1 | 0.421 |

Data were expressed as mean ± SEM.
*denotes statistically significant difference between groups (p < 0.05).
Abbreviations: AUC: area under the concentration-time curve; Cₘₐₓ: maximal concentration; Tₘₐₓ: time to maximal concentration; T_{1/2}: elimination half-life; C_{trough}: trough level at day 14; BMI: body mass index

The study has been conducted according to applicable US federal regulations and institutional policies (which are based in federal regulations, guidance, and ICH Good Clinical Practice guidelines) after approval of the protocol and any amendments by the Boston Medical Center and Boston University Medical Campus IRB. If in the course of the study unanticipated problems, safety monitor's reports and adverse events occurred they were reported in line with the internal regulations at BMC/BU Medical Center IRB in line with the general IRB policies applicable for such studies. Where necessary appropriate steps were taken to ensure the well-being, safety and integrity of the participating subjects. Data handling and record keeping was made in line internal regulations at BMC/BU Medical Center taking into account the corresponding government regulations.

Based on the above clinical study the inventors come to the following conclusions and recommendations in connection with the treatment of vitamin D deficiencies:
Obese (or overweight) persons or persons with a respective genetic disposition to get obese need more vitamin D than “normal ones”.
It is known that when the high amounts of vitamin D they need to get a reasonable blood level of 25(OH)D are administered to such persons most of the vitamin D ends up in their fatty tissues. While such heavy load does not manifest itself in clearly observable systemic toxic effects, it cannot be excluded that some negative or counterproductive effects may occur locally (e.g. in the fatty tissue).
In addition, the uptake of vitamin D from lymph to blood is probably reduced in obese persons, persons having the tendency to get obese and in persons having a history of malabsorption of vitamin D or persons with a genetic disposition to develop obesity.
Based on the above it would seem logic to just supplement 25(OH)D to such persons. Quite clear by now is that an approximate blood level of 25(OH)D>30 ng/mL should be targeted. However, there is an aspect which is a priori unrelated to the above, namely that there is increasing evidence that vitamin D per se may have additional favourable biological activities (e.g. in stabilizing cell membranes) besides the fact that in general it is metabolized in the liver to 25(OH)D and then to the vital 1,25(OH)2D in the kidney. The latter metabolite is known to regulate more than 700 genomically controlled processes.
This growing evidence of favourable effects of vitamin D3 and D2 per se puts a question mark behind supplementing just 25(OH)D to obese who are in the average anyway having less vitamin D3 and D2 per se in their blood than people with lower BMI.
Therefore, if just this aspect is considered, it would appear to be logical to supplement even more vitamin D to obese than to non-obese.
The two above groups of arguments seem to be directed to contradictory solutions of the problem to treat vitamin D deficiencies, particularly in obese persons, overweight persons having the tendency to get obese or persons having a history of malabsorption of vitamin D or persons with a genetic disposition to develop obesity. One solution points to administering as little vitamin D3 and D2 per se as possible. The other solution points to administering by all means not too little vitamin D3 and D2 per se.
In contrast to the case of 25(OH)D the ideal “blood level” of vitamin D3 and/or D2 per se, is in essence still unknown. The reason is, e.g., that there is much less of a constant “level” of vitamin D3 and D2 per se in comparison to 25(OH)D, because the former has a much shorter T_{1/2} than the latter and in addition less affinity to the binding protein (DBP) in the blood and a higher tendency to disappear in the fat—besides the fact that its concentration is in addition lower in the average. Accordingly, one consideration is that periodic peaks of vitamin D3 and D2 per se are what the organism needs. The other consideration is that one has to take into account that the skin, where vitamin D3 is naturally formed, can actually be regarded like a slow release compartment.
Finally, a point regarding analytics: One is routinely analysing 25(OH)D in blood, because it is less “jumpy” than vitamin D3 and D2 per se, the average concentration is higher, and the assay is well established. Nevertheless, it is also possible to measure vitamin D3 and D2 per se in serum as it is described above.
The above conclusions point towards the solution that vitamin D insufficiencies or deficiencies, particularly in obese persons, overweight persons having the tendency to get obese or persons having a history of malabsorption of vitamin D or persons with a genetic disposition to develop obesity should be treated with (i) a combination of 25(OH)D3 and/or 25(OH)D2 and vitamin D2; or (ii) a combination of 25(OH)D3 and/or 25(OH)D2 and vitamin D3; or (iii) a combination of 25(OH)D3 and/or 25(OH)D2 and a combination of vitamin D2 and vitamin D3 in a BMI-dependent way, whereby also lifestyle factors and/or diet factors are to be considered. Some people may not need the added vitamin D3 and D2 per se, because of their lifestyle provides them with sufficient vitamin D production in the skin and/or their dietary uptake of vitamin D provides them a sufficient level of vitamin D. Another point to take into account is that some obese persons may a priori have a comparatively high blood level of 25(OH)D and correspondingly do need less or no supplementation of 25(OH)D: Other obese persons who are low on 25(OH)D need a corresponding amount of 25(OH)D being supplemented. In cases where an obese person is just slightly low in 25(OH)D supplementing 25(OH)D alone might do, because this “comparatively high level” of 25(OH)D is indicating that vitamin D3 and D2 per se won't be very low either. In rare cases the 25(OH)D level is in the ideal range and thus no 25(OH)D must be supplemented.

1. A composition comprising a vitamin D supplement for use in the treatment of and/or prophylaxis of vitamin D insufficiency or deficiency in obese persons, wherein the composition comprises a unit dose of vitamin D and 25(OH)D in a dosage for daily administration selected from the group as follows:
(i) for individuals of BMI of 30 to 36 of unknown or unreliably known level of 25(OH)D in blood, respectively, who have a normal lifestyle with respect to UV-light exposure and daily uptake of vitamin D, the dose comprises vitamin D and 25(OH)D in a ratio of 1 to 1 and in amounts at the lower end of the range of 5 to 7.5 μg of each of the vitamin D and the 25(OH)D, wherein the lower end of the range means to encompass the range from the lowest end of the range up to the middle of the specified range;
(ii) for individuals of BMI of 30 to 36 of unknown or unreliably known level of 25(OH)D in blood, respectively, who have an unfavorable lifestyle with respect to UV-light exposure and daily uptake of vitamin D, the dose comprises vitamin D and 25(OH)D in a ratio of 1 to 1 and in amounts at the higher end of the range of 5 to 7.5 μg of each of the vitamin D and the 25(OH)D, wherein the higher end of the range means to encompass the range from the middle of the range up to the highest end of the specified range;
(iii) for individuals of BMI of 30 to 36 of reliably known level of 25(OH)D in blood of >20 to 30 ng/mL the dose comprises vitamin D and 25(OH)D in a ratio of 1 to 1 and in amounts in the range of 5 to 7.5 μg of each of the vitamin D and the 25(OH)D;
(iv) for individuals of BMI of 30 to 36 of reliably known level of 25(OH)D in blood of >10 to 20 ng/mL the dose comprises vitamin D and 25(OH)D in a ratio of 1 to 1.5, with the dose of vitamin D at 5 to 7.5 μg and the dose of 25(OH)D at 7.5 to 11 μg;
(v) for individuals of BMI of 30 to 36 of reliably known level of 25(OH)D in blood of ≤10 ng/mL the dose comprises vitamin D and 25(OH)D in a ratio of 1 to 2, with the dose of vitamin D at 5 to 7.5 μg and the dose of 25(OH)D at 10 to 15 μg;
(vi) for individuals of BMI of 37 to 43 of unknown or unreliably known level of 25(OH)D in the blood, respectively, who have a normal lifestyle with respect to UV-light exposure and daily uptake of vitamin D, the dose comprises vitamin D and 25(OH)D in a ratio of 1 to 1 and in amounts at the lower end of the range of 7.5 to 11 μg of each of the vitamin D and the 25(OH)D, wherein the lower end of the range means to encompass the range from the lowest end of the range up to the middle of the specified range;
(vii) for individuals of BMI of 37 to 43 of unknown or unreliably known level of 25(OH)D in the blood, respectively, who have an unfavorable lifestyle with respect to UV-light exposure and daily uptake of vitamin D, the dose comprises vitamin D and 25(OH)D in a ratio of 1 to 1 and in amounts at the higher end of the range of 7.5 to 11 μg of each of vitamin D and the 25(OH)D, wherein the higher end of the range means to encompass the range from the middle of the range up to the highest end of the specified range;
(viii) for individuals of BMI of 37 to 43 of reliably known level of 25(OH)D in blood of >20 to 30 ng/mL the dose comprises vitamin D and 25(OH)D in a ratio of 1.5 to 1, with the dose of vitamin D at 7.5 to 11 μg, and a dose of 25(OH)D of 5 to 7.5 μg;
(ix) for individuals of BMI of 37 to 43 of reliably known level of 25(OH)D in blood of >10 to 20 ng/mL the dose comprises vitamin D and 25(OH)D in a ratio of 1 to 1 and in amounts of 7.5 to 11 μg of each of the two products;
(x) for individuals of BMI of 37 to 43 of reliably known level of 25(OH)D in blood of ≤10 ng/mL the dose comprises vitamin D and 25(OH)D in a ratio of 1 to 1.5, with the dose of vitamin D at 7.5 to 11 μg, and a dose of 25(OH)D of 11 to 17 μg;
(xi) for individuals of BMI of more than 43 of unknown or unreliably known level of 25(OH)D in blood, respectively, who have a normal lifestyle with respect to UV-light exposure and daily uptake of vitamin D, the dose comprises vitamin D and 25(OH)D in a ratio of 1 to 1 and in amounts at the lower end of the range of 11 to 15 μg of each of the two products, wherein the lower end of the range means to encompass the range from the lowest end of the range up to the middle of the specified range;
(xii) for individuals of BMI of more than 43 of unknown or unreliably known level of 25(OH)D in blood, respectively, who have an unfavorable lifestyle with respect to UV-light exposure and daily uptake of vitamin D, the dose comprises vitamin D and 25(OH)D in a ratio of 1 to 1 and in amounts at the higher end of the range of 11 to 15 μg of each of the vitamin D and the 25(OH)D, wherein the higher end of the range means to encompass the range from the middle of the range up to the highest end of the specified range;
(xiii) for individuals of BMI of more than 43 of reliably known level of 25(OH)D in blood of >20 to 30 ng/mL the dose comprises vitamin D and 25(OH)D in a ratio of 1.5 to 1, with the dose of vitamin D at 11 to 15 μg, and a dose of 25(OH)D of 7.5 to 10 μg;
(xiv) for individuals of BMI of more than 43 of reliably known level of 25(OH)D in blood of >10 to 20 ng/mL the dose comprises vitamin D and 25(OH)D in a ratio of 1 to 1 and in amounts of 11 to 15 μg of each of the vitamin D and the 25(OH)D; or
(xv) for individuals of BMI of more than 43 of reliably known level of 25(OH)D in blood of ≤10 ng/mL the dose comprises vitamin D and 25(OH)D in a ratio of 1 to 1.5, with the dose of vitamin D at 11 to 15 μg, and a dose of 25(OH)D of 17 to 22.5 μg.

2. The composition according to claim 1, wherein for individuals of BMI of 30 to 36 of reliably known level of 25(OH)D in blood of >20 to 30 ng/mL the dose comprises vitamin D and 25(OH)D in a ratio of 1 to 1 and in amounts in the range of 5 to 7.5 μg of each of the vitamin D and the 25(OH)D.

3. The composition according to claim 1, wherein for individuals of BMI of 30 to 36 of reliably known level of 25(OH)D in blood of >10 to 20 ng/mL the dose comprises vitamin D and 25(OH)D in a ratio of 1 to 1.5, with the dose of vitamin D at 5 to 7.5 μg and the dose of 25(OH)D at 7.5 to 11 μg.

4. The composition according to claim 1, wherein for individuals of BMI of 30 to 36 of reliably known level of 25(OH)D in blood of ≤10 ng/mL the dose comprises vitamin D and 25(OH)D in a ratio of 1 to 2, with the dose of vitamin D at 5 to 7.5 μg the dose of 25(OH)D at 10 to 15 μg.

5. The composition according to claim 1, wherein for individuals of BMI of 37 to 43 of reliably known level of 25(OH)D in blood of >20 to 30 ng/mL the dose comprises vitamin D and 25(OH)D in a ratio of 1.5 to 1, with the dose of vitamin D at 7.5 to 11 μg, and a dose of 25(OH)D of 5 to 7.5 μg.

6. The composition according to claim 1, wherein for individuals of BMI of 37 to 43 of reliably known level of 25(OH)D in blood of >10 to 20 ng/mL the dose comprises vitamin D and 25(OH)D in a ratio of 1 to 1 and in amounts of 7.5 to 11 μg of each of the vitamin D and the 25(OH)D.

7. The composition according to claim 1, wherein for individuals of BMI of 37 to 43 of reliably known level of 25(OH)D in blood of ≤10 ng/mL the dose comprises vitamin D and 25(OH)D in a ratio of 1 to 1.5, with the dose of vitamin D at 7.5 to 11 μg, and a dose of 25(OH)D of 11 to 17 μg.

8. The composition according to claim 1, wherein for individuals of BMI of more than 43 of reliably known level of 25(OH)D in blood of >20 to 30 ng/mL the dose comprises vitamin D and 25(OH)D in a ratio of 1.5 to 1, with the dose of vitamin D at 11 to 15 μg, and a dose of 25(OH)D of 7.5 to 10 μg.

9. The composition according to claim 1, wherein for individuals of BMI of more than 43 of reliably known level of 25(OH)D in blood of >10 to 20 ng/mL the dose comprises vitamin D and 25(OH)D in a ratio of 1 to 1 and in amounts of 11 to 15 μg of each of the vitamin D and the 25(OH)D.

10. The composition according to claim 1, wherein for individuals of BMI of more than 43 of reliably known level of 25(OH)D in blood of ≤10 ng/mL the dose comprises vitamin D and 25(OH)D in a ratio of 1 to 1.5, with the dose of vitamin D at 11 to 15 μg, and a dose of 25(OH)D of 17 to 22.5 μg.

11. The composition according to claim 1, wherein 25(OH)D is 25(OH)D3 and vitamin D is vitamin D3.
